SEQUENCE LISTING (7 sequences)

SEQ ID NO: 1 - protein, 140 aa, Homo sapiens
MRSPRMRVCAKSVLLSHWLFLAYVLMVCCKLMSASSQHLRGHAGHHQIKQGTCEVVAVHRCCNKNRIEERSQTVKCSCFPGQVAGTTRAQPSCVEASIVIQKWWCHMNPCLEGEDCKVLPDYSGWSCSSGNKVKTTKVTR

SEQ ID NO: 2 - protein, 169 aa, Homo sapiens
MRSPRMRVCAKSVLLSHWLFLAYVLMVCCKLMSASSQHLRGHAGHHQIKQGTCEVVAVHRCCNKNRIEERSQTVKCSCFPGQVAGTTRAQPSCVEASIVIQKWWCHMNPCLEGEDCKVLPDYSGNAVAKREVCFNPGGAAGGGALLLGFPSWPLCRKLSRISATSYLYM

SEQ ID NO: 3 - RNA, 2274 nt, Homo sapiens
  source 1..2274: mol_type = mRNA
aattttcgggtcgacgatttcgtgggctcggctccgcactgctagctgcgcgccgccctggacggggcgcaccgactgcgcgcgcggctgcgggcaaacatcgggagtcctgcctcagctgccgcttctccagcagcagcttcaggcttctcccgcaggagcttcgggcttctcctggtagagacgtgggaacttttcttctcctggcgaggctgcagaggtgatgggccgctcccggggctcccgcggggaggcggcacggtgagcgtcctcgggctccggtgcggcgatcagtacctagttccggacgcgccggtccgacttggatgccggctctagtcgagtcttctgagctacgataattttttggaacggcagaaatgattggttctagcaacagatgggaatttggagtcactctgaaatatatcctggaataagtgtgtttgactagaaccacatcttatgaggtccccaaggatgagagtctgtgctaagtcagtgttgctgtcgcactggctctttctagcctacgtgttaatggtgtgctgtaagctgatgtccgcctcaagccagcacctccggggacatgcaggtcaccaccaaatcaagcaagggacctgtgaggtggtcgccgtgcacaggtgctgcaataagaaccgcatagaagagcggtcacaaacggtcaagtgctcttgcttcccgggacaggtggcgggcacaactcgggctcaaccttcttgtgttgaagcttccattgtgattcagaaatggtggtgtcacatgaatccgtgtttggaaggagaggattgtaaagtgctgccagattactcaggttggtcctgtagcagtggcaataaagtcaaaactacgaaggtaacgcggtagcgaagagagaggtgtgcttcaatcctggaggggcagcaggaggcggagctcttttgcttggattcccatacatggcccctctgcagaaaattgtctaggatttcagcaacttcatatttgtatattgtgagctgtgagaaggtggcattcacttaactggcccagccctctctgcttcgtgattttatttcattgaattataaccacaagccacaacccatttgacatcctctctcggattcccaaggagcatacctccaaaatccgagaagagcaaatcagagtcttcaaaatggatcaccactaagggcatgttcattcttcactttctttctgcttttacaaaagaacttggatgtatgttccaaagggtcctcattctgttcctcttttgaacttttccttttgtccttgtattaaagtggttttaaaggggtctaaaaagattttggcaaaacatatttgcagatgtagattagctggtgaagaaaattactgctagagatcaactgattaactggtaaagaacgtttattttataacccttgaagaatagaaggacatagttggattattgtgtgtgcattgtatttttacttctattttttttttgctttccattttccagttagcagagataaaatgagagcgttttaacttcaatgtaccattttactgagtgctaaggaagcatatcaattccaatattttataaccaaagctctatcagaacatatttataaaacttgttggaatttttacggctttggtgtagtcatgtaggtaaatcatttaaaatataaaacaatctcaatttagatcaagggttatttcttagatcaaatttatgccaattatatgaaaagattttaactccgagacaggagtctttcagtgctgaatttttagactgtaaatgagttcttcttaacttagctgtttccctacttctgtgacttctgtgttagccatcttatttctttaaaatctgagtcctgattggcttaatgattttgcagcagacatgtctccacatattctcaaatgctgtcatgcggaaacgtatgaaacagatgaagaatgactgacccagattttagatgtataatgttgttaaagtacatactactgtaaaaatatgggatgaattttatatattaagaaatgccaaaaacatagtttctgcaccaagttaattatccctgtcctttcacatttatagggggaaaataaatactttatagcctaacagttatttgatgttattcttgcagagggaatggaaaggaatggaaagatttgttggcgtaatttttgaatatttgttatgatcatatgaataagtaaaaaaattcatcctgctgatggcaaaaaaaaaaaaaaaaa

SEQ ID NO: 4 - RNA, 2229 nt, Homo sapiens
  source 1..2229: mol_type = mRNA
gcactgctagctgcgcgccgccctggacggggcgcaccgactgcgcgcgcggctgcgggcaaacatcgggagtcctgcctcagctgccgcttctccagcagcagcttcaggcttctcccgcaggagcttcgggcttctcctggtagagacgtgggaacttttcttctcctggcgaggctgcagaggtgatgggccgctcccggggctcccgcggggaggcggcacggtgagcgtcctcgggctccggtgcggcgatcagtacctagttccggacgcgccggtccgacttggatgccggctctagtcgagtcttctgagctacgataattttttggaacggcagaaatgattggttctagcaacagatgggaatttggagtcactctgaaatatatcctggaataagtgtgtttgactagaaccacatcttatgaggtccccaaggatgagagtctgtgctaagtcagtgttgctgtcgcactggctctttctagcctacgtgttaatggtgtgctgtaagctgatgtccgcctcaagccagcacctccggggacatgcaggtcaccaccaaatcaagcaagggacctgtgaggtggtcgccgtgcacaggtgctgcaataagaaccgcatagaagagcggtcacaaacggtcaagtgctcttgcttcccgggacaggtggcgggcacaactcgggctcaaccttcttgtgttgaagcttccattgtgattcagaaatggtggtgtcacatgaatccgtgtttggaaggagaggattgtaaagtgctgccagattactcaggttggtcctgtagcagtggcaataaagtcaaaactacgaaggtaacgcggtagcgaagagagaggtgtgcttcaatcctggaggggcagcaggaggcggagctcttttgcttggattcccatcatggcccctttgcagaaaattgtctaggatttcagcaacttcatatttgtatatgtgagctgtgagaggtggcattcacttaactggcccagccctctctgcttcgtgattttatttcattgaattataaccacaagccaccacccatttgacatcctctctggattcccaaggagcatacctccaaaatccgagaagagcaaatcagagtcttcaaaatggatcaccactaagggcatgttcattcttcactttctttctgcttttacaaaagaacttggatgtatgttccaaagggtcctcattctgttcctcttttgaacttttccttttgtccttgtattaaagtggttttaaaggggtctaaaaagattttggcaaaacatatttgcagatgtagattagctggtgaagaaaattactgctagagatcaactgattaactggtaaagaacgtttattttataacccttgaagaatagaaggacatagttggattattgtgtgtgcattgtatttttacttctattttttttttgctttccattttccagttagcagagataaaatgagagcgttttaacttcaatgtaccattttactgagtgctaaggaagcatatcaattccaatattttataaccaaagctctatcagaacatatttataaaacttgttggaatttttacggcttttgtgtagtcatgtaggtaaatcatttaaaatataaaacaatctcaatttagatcaagggttatttcttagatcaaatttatgccaattatatgaaaagattttaactccgagacaggagtctttcagtgctgaatttttagactgtaaatgagttcttcttaacttagctgtttccctacttctgtgacttctgtgttagccatcttatttctttaaaatctgagtcctgattggcttaatgattttgcagcagacatgtctccacatattctcaaatgctgtcatgcggaaacgtatgaaacagatgaagaatgactgacccagattttagatgtataatgttgttaaagtacatactactgtaaaaatatgggatgaattttatatattaagaaatgccaaaaacatagtttctgcaccaagttaattatccctgtcctttcacatttatagggggaaaataaatactttaatgttgtttatagcctaacagttatttgattttattcttgcagagggaatggaaaggaatggaaagatttgttggcgtaatttttgaatatttgttatgatcatatgaataagtaaaaaaattcatcctgctgatggca

SEQ ID NO: 5 - RNA, 2147 nt, Homo sapiens
  source 1..2147: mol_type = mRNA
gcactgctagctgcgcgccgccctggacggggcgcaccgactgcgcgcgcggctgcgggcaaacatcgggagtcctgcctcagctgccgcttctccagcagcagcttcaggcttctcccgcaggagcttcgggcttctcctggtagagacgtgggaacttttcttctcctggcgaggctgcagaggtgatgggccgctcccggggctcccgcggggaggcggcacggtcttctgagctacgataattttttggaacggcagaaatgattggttctagcaacagatgggaatttggagtcactctgaaatatatcctggaataagtgtgtttgactagaaccacatcttatgaggtccccaaggatgagagtctgtgctaagtcagtgttgctgtcgcactggctctttctagcctacgtgttaatggtgtgctgtaagctgatgtccgcctcaagccagcacctccggggacatgcaggtcaccaccaaatcaagcaagggacctgtgaggtggtcgccgtgcacaggtgctgcaataagaaccgcatagaagagcggtcacaaacggtcaagtgctcttgcttcccgggacaggtggcgggcacaactcgggctcaaccttcttgtgttgaagcttccattgtgattcagaaatggtggtgtcacatgaatccgtgtttggaaggagaggattgtaaagtgctgccagattactcaggttggtcctgtagcagtggcaataaagtcaaaactacgaaggtaacgcggtagcgaagagagaggtgtgcttcaatcctggaggggcagcaggaggcggagctcttttgcttggattcccatcatggcccctttgcagaaaattgtctaggatttcagcaacttcatatttgtatatgtgagctgtgagaggtggcattcacttaactggcccagccctctctgcttcgtgattttatttcattgaattataaccacaagccaccacccatttgacatcctctctggattcccaaggagcatacctccaaaatccgagaagagcaaatcagagtcttcaaaatggatcaccactaagggcatgttcattcttcactttctttctgcttttacaaaagaacttggatgtatgttccaaagggtcctcattctgttcctcttttgaacttttccttttgtccttgtattaaagtggttttaaaggggtctaaaaagattttggcaaaacatatttgcagatgtagattagctggtgaagaaaattactgctagagatcaactgattaactggtaaagaacgtttattttataacccttgaagaatagaaggacatagttggattattgtgtgtgcattgtatttttacttctattttttttttgctttccattttccagttagcagagataaaatgagagcgttttaacttcaatgtaccattttactgagtgctaaggaagcatatcaattccaatattttataaccaaagctctatcagaacatatttataaaacttgttggaatttttacggcttttgtgtagtcatgtaggtaaatcatttaaaatataaaacaatctcaatttagatcaagggttatttcttagatcaaatttatgccaattatatgaaaagattttaactccgagacaggagtctttcagtgctgaatttttagactgtaaatgagttcttcttaacttagctgtttccctacttctgtgacttctgtgttagccatcttatttctttaaaatctgagtcctgattggcttaatgattttgcagcagacatgtctccacatattctcaaatgctgtcatgcggaaacgtatgaaacagatgaagaatgactgacccagattttagatgtataatgttgttaaagtacatactactgtaaaaatatgggatgaattttatatattaagaaatgccaaaaacatagtttctgcaccaagttaattatccctgtcctttcacatttatagggggaaaataaatactttaatgttgtttatagcctaacagttatttgattttattcttgcagagggaatggaaaggaatggaaagatttgttggcgtaatttttgaatatttgttatgatcatatgaataagtaaaaaaattcatcctgctgatggca

SEQ ID NO: 6 - RNA, 2106 nt, Homo sapiens
  source 1..2106: mol_type = mRNA
gcactgctagctgcgcgccgccctggacggggcgcaccgactgcgcgcgcggctgcgggcaaacatcgggagtcctgcctcagctgccgcttctccagcagcagcttcaggcttctcccgcaggagcttcgggcttctcctggtagagacgtgggaacttttcttctcctggcgaggctgcagaggtgatgggccgctcccggggctcccgcggggaggcggcacggtcttctgagctacgataattttttggaacggcagaaatgattggttctagcaacagatgggaatttggagtcactctgaaatatatcctggaataagtgtgtttgactagaaccacatcttatgaggtccccaaggatgagagtctgtgctaagtcagtgttgctgtcgcactggctctttctagcctacgtgttaatggtgtgctgtaagctgatgtccgcctcaagccagcacctccggggacatgcaggtcaccaccaaatcaagcaagggacctgtgaggtggtcgccgtgcacaggtgctgcaataagaaccgcatagaagagcggtcacaaacggtcaagtgctcttgcttcccgggacaggtggcgggcacaactcgggctcaaccttcttgtgttgaagcttccattgtgattcagaaatggtggtgtcacatgaatccgtgtttggaaggagaggattgtaaagtgctgccagattactcaggtaacgcggtagcgaagagagaggtgtgcttcaatcctggaggggcagcaggaggcggagctcttttgcttggattcccatcatggcccctttgcagaaaattgtctaggatttcagcaacttcatatttgtatatgtgagctgtgagaggtggcattcacttaactggcccagccctctctgcttcgtgattttatttcattgaattataaccacaagccaccacccatttgacatcctctctggattcccaaggagcatacctccaaaatccgagaagagcaaatcagagtcttcaaaatggatcaccactaagggcatgttcattcttcactttctttctgcttttacaaaagaacttggatgtatgttccaaagggtcctcattctgttcctcttttgaacttttccttttgtccttgtattaaagtggttttaaaggggtctaaaaagattttggcaaaacatatttgcagatgtagattagctggtgaagaaaattactgctagagatcaactgattaactggtaaagaacgtttattttataacccttgaagaatagaaggacatagttggattattgtgtgtgcattgtatttttacttctattttttttttgctttccattttccagttagcagagataaaatgagagcgttttaacttcaatgtaccattttactgagtgctaaggaagcatatcaattccaatattttataaccaaagctctatcagaacatatttataaaacttgttggaatttttacggcttttgtgtagtcatgtaggtaaatcatttaaaatataaaacaatctcaatttagatcaagggttatttcttagatcaaatttatgccaattatatgaaaagattttaactccgagacaggagtctttcagtgctgaatttttagactgtaaatgagttcttcttaacttagctgtttccctacttctgtgacttctgtgttagccatcttatttctttaaaatctgagtcctgattggcttaatgattttgcagcagacatgtctccacatattctcaaatgctgtcatgcggaaacgtatgaaacagatgaagaatgactgacccagattttagatgtataatgttgttaaagtacatactactgtaaaaatatgggatgaattttatatattaagaaatgccaaaaacatagtttctgcaccaagttaattatccctgtcctttcacatttatagggggaaaataaatactttaatgttgtttatagcctaacagttatttgattttattcttgcagagggaatggaaaggaatggaaagatttgttggcgtaatttttgaatatttgttatgatcatatgaataagtaaaaaaattcatcctgctgatggca

SEQ ID NO: 7 - RNA, 2188 nt, Homo sapiens
  source 1..2188: mol_type = mRNA
gcactgctagctgcgcgccgccctggacggggcgcaccgactgcgcgcgcggctgcgggcaaacatcgggagtcctgcctcagctgccgcttctccagcagcagcttcaggcttctcccgcaggagcttcgggcttctcctggtagagacgtgggaacttttcttctcctggcgaggctgcagaggtgatgggccgctcccggggctcccgcggggaggcggcacggtgagcgtcctcgggctccggtgcggcgatcagtacctagttccggacgcgccggtccgacttggatgccggctctagtcgagtcttctgagctacgataattttttggaacggcagaaatgattggttctagcaacagatgggaatttggagtcactctgaaatatatcctggaataagtgtgtttgactagaaccacatcttatgaggtccccaaggatgagagtctgtgctaagtcagtgttgctgtcgcactggctctttctagcctacgtgttaatggtgtgctgtaagctgatgtccgcctcaagccagcacctccggggacatgcaggtcaccaccaaatcaagcaagggacctgtgaggtggtcgccgtgcacaggtgctgcaataagaaccgcatagaagagcggtcacaaacggtcaagtgctcttgcttcccgggacaggtggcgggcacaactcgggctcaaccttcttgtgttgaagcttccattgtgattcagaaatggtggtgtcacatgaatccgtgtttggaaggagaggattgtaaagtgctgccagattactcaggtaacgcggtagcgaagagagaggtgtgcttcaatcctggaggggcagcaggaggcggagctcttttgcttggattcccatcatggcccctttgcagaaaattgtctaggatttcagcaacttcatatttgtatatgtgagctgtgagaggtggcattcacttaactggcccagccctctctgcttcgtgattttatttcattgaattataaccacaagccaccacccatttgacatcctctctggattcccaaggagcatacctccaaaatccgagaagagcaaatcagagtcttcaaaatggatcaccactaagggcatgttcattcttcactttctttctgcttttacaaaagaacttggatgtatgttccaaagggtcctcattctgttcctcttttgaacttttccttttgtccttgtattaaagtggttttaaaggggtctaaaaagattttggcaaaacatatttgcagatgtagattagctggtgaagaaaattactgctagagatcaactgattaactggtaaagaacgtttattttataacccttgaagaatagaaggacatagttggattattgtgtgtgcattgtatttttacttctattttttttttgctttccattttccagttagcagagataaaatgagagcgttttaacttcaatgtaccattttactgagtgctaaggaagcatatcaattccaatattttataaccaaagctctatcagaacatatttataaaacttgttggaatttttacggcttttgtgtagtcatgtaggtaaatcatttaaaatataaaacaatctcaatttagatcaagggttatttcttagatcaaatttatgccaattatatgaaaagattttaactccgagacaggagtctttcagtgctgaatttttagactgtaaatgagttcttcttaacttagctgtttccctacttctgtgacttctgtgttagccatcttatttctttaaaatctgagtcctgattggcttaatgattttgcagcagacatgtctccacatattctcaaatgctgtcatgcggaaacgtatgaaacagatgaagaatgactgacccagattttagatgtataatgttgttaaagtacatactactgtaaaaatatgggatgaattttatatattaagaaatgccaaaaacatagtttctgcaccaagttaattatccctgtcctttcacatttatagggggaaaataaatactttaatgttgtttatagcctaacagttatttgattttattcttgcagagggaatggaaaggaatggaaagatttgttggcgtaatttttgaatatttgttatgatcatatgaataagtaaaaaaattcatcctgctgatggca

(19) United States
(12) Patent Application Publication
(10) Pub. No.: US 2026/0235581 A1
(43) Pub. Date: Aug. 13, 2026

(54) METHOD FOR THE SELECTION OF AN ECOBIOLOGICAL COMPOUND CAPABLE OF INCREASING THE SKIN TOLERANCE THRESHOLD

(71) Applicants: Jean-Noël THOREL, PARIS (FR); NAOS INSTITUTE OF LIFE SCIENCE, AIX-EN-PROVENCE (FR)

(72) Inventor: Jean-Noël THOREL, PARIS (FR)

(21) Appl. No.: 19/153,470

(22) PCT Filed: Feb. 19, 2024
(86) PCT No.: PCT/EP2024/054201

§ 371 Date: Aug. 4, 2025

(30) Foreign Application Priority Data
Feb. 17, 2023 (EP) 23157371.8

(51) Int. Cl. G01N 33/50; A61K 8/36; A61K 8/64; A61K 8/73; A61Q 19/00; C12Q 1/6876

(52) U.S. Cl. CPC G01N 33/5044; A61K 8/361; A61K 8/64; A61K 8/73; A61Q 19/00; C12Q 1/6876; G01N 33/5055; G01N 33/5058; C12Q 2600/148; C12Q 2600/158; G01N 2333/521

(57) ABSTRACT

A method for the selection of at least one compound, preferably ecobiological, is provided, intended for the prevention and/or care of sensitive and/or sensitised skin, implementing the identification of a modulation of the content of TAFA4 and/or of nucleic acid encoding it.

### FIELD OF THE INVENTION

The present invention relates to a method for selecting at least one compound capable of increasing the skin tolerance threshold, in particular via the activity of the chemokine TAFA4. In particular, this is a method for selecting at least one compound, preferably ecobiological, intended for the prevention and/or care of sensitive and/or sensitised skin, comprising, in particular, a step of quantifying the content of TAFA4, and/or of nucleic acids encoding TAFA4, and a step of comparing this quantification with a reference value so as to identify the presence or not of a variation of the content of TAFA4 and/or of nucleic acids encoding TAFA4.

### PRIOR ART

The skin constitutes the main barrier which separates the organism from the external environment and is composed of the superposition of three main layers, respectively from the deepest to the most superficial: the hypodermis, the dermis and the epidermis, colonised by the microbiota, which is often considered as the ultimate skin layer. Due to its constant interaction with the environment, the skin suffers multiple attacks which alter its balance. In parallel with this exogenous harm, each individual has internal factors having an impact on the health of the skin, namely the genetic makeup, or also the hormones. These different exogenous and endogenous factors cause remarkable differences between individuals in the levels of skin tolerance and sensitivity, with an increasing part of the population which perceives their skin as irritable or sensitive.

The results of epidemiological studies show that sensitive skin syndrome is a global problem, which is constantly increasing with a prevalence which varies from one country to another (Misery et al., 2018b). In France, 59% of the population declares to have sensitive skin (Misery et al., 2018a). More generally, in Europe in recent years, the prevalence of sensitive skin has increased considerably, namely by +7% in 10 years (Misery et al., 2018a). Comparable prevalences have also been found in Asia and in Latin America.

Sensitive skin is characterised by skin hyperresponsiveness: the skin reacts excessively to external and internal attacks and to stimuli which should normally be well-tolerated (Ständer et al., 2009). In other words, sensitive skin reacts more rapidly, due to a lowering of its tolerance threshold.

Sensitive skin is now recognised as a real syndrome by the medical community with an official definition established by the International Forum for the Study of Itch (IFSI): “syndrome defined by the occurrence of unpleasant sensations (tingling, tightness, heating-up, itching and irritation) in response to stimuli which should not normally cause such sensations. The skin can appear normal or be accompanied by erythema. Sensitive skin can affect all parts of the body, in particular, the face” (Misery et al., 2017).

According to the current model accepted by the scientific community, sensitive skin syndrome is the result of a cumulative effect of a set of skin changes described below:
an altered barrier function, which makes it easier for aggressive agents to penetrate the skin, resulting in skin inflammation;
an alteration of the cutaneous nerve endings of type C, in particular a neuropathy of the small nerve fibres (Huet et al., 2018);
non-specific inflammation of keratinocytes involving pro-inflammatory mediators such as cytokines (IL-1, IL-8, TNFα), inflammatory lipid mediators (E2 prostaglandins, F2α prostaglandins and leukotrienes) and histamine; and
neurogenic inflammation generated by the release of neuromediators, which maintain skin inflammation and cause vasodilation (Ständer et al., 2009).

Skin hypersensitivity has a considerable impact on the overall quality of life and often induces people with this condition to reduce or stop the use of cosmetics in order not to exacerbate irritation. Indeed, cosmetic products generally contain compounds which can cause inflammatory phenomena, such as surfactants or preservatives. These intolerance reactions can be specific to an individual and limited to very specific conditions and/or combinations of compounds, and are therefore difficult to predict. Yet, in the case of specific products such as sunscreen products and products targeting particular dermatological conditions, reducing the frequency of application and/or the quantities of cosmetic products applied is not recommended at the risk of leading to an aggravation of skin hypersensitivity.

Moreover, it is important to be able to control skin sensitivity, for example, that generated by atmospheric agents (pollution, temperature, humidity, etc.), allergy phenomena, healing or also sunburn by using non-pharmaceutical cosmetic compounds, advantageously ecobiological, in particular presented in a form capable of a topical application.

As an example, document EP 1 457 780 describes a screening method, in which the decrease in the production of stem cell factor (SCF), used as a specific biomarker, enables the selection of an ingredient of interest capable of treating pruritus and dry or sensitive skin syndromes. However, SCF is known at the cutaneous level to be mainly involved in the survival of melanocytes and in the induction of melanin synthesis (Grabbe et al., 1994; Atef et al., 2019). Consequently, modulation of this factor could lead to significant adverse effects in terms of skin pigmentation.

Therefore, there remains a clear need for a method for identifying compounds, preferably non-pharmaceutical cosmetic compounds, in particular capable of being presented in a galenic form, adapted to a topical and/or oral administration, advantageously ecobiological, which make it possible to increase the skin tolerance threshold and/or reduce skin discomfort, also by acting on the endogenous mechanisms for controlling pain and inflammation specific to human skin.

To date, the TAFA4 (TAFA Chemokine Like Family Member 4) protein, also called FAM19A4 (family with sequence similarity 19 (chemokine (C—C motif)-like) member A4), a neurokine mainly produced in humans by certain nerve fibres (Wang et al., 2015; Yoo et al., 2021), has been identified as an anti-inflammatory and soothing agent. In humans, the amino acid sequence of TAFA4 corresponds to the sequence SEQ ID NO: 1 and/or to the sequence SEQ ID NO: 2 and/or is encoded by the nucleotide sequence of the mRNAs corresponding, partially or totally, to the sequence SEQ ID NO: 3, SEQ ID NO: 4, SEQ ID NO: 5, SEQ ID NO: 6 or SEQ ID NO: 7.

The TAFA4 protein is composed of two domains, including a signal peptide and a highly preserved chemokine-like sequence (Wang et al., 2015). In humans, TAFA4 is strongly expressed in the brain, and more weakly in other organs such as the colon, spleen, pancreas, prostate, liver or also lungs (Leeman et al., 2020; Liu et al., 2017; Tom Tang et al., 2004; Wang et al., 2015).

At the cutaneous level, in vivo in mice, TAFA4 is expressed and secreted by certain neurons, the free endings of which are found in the skin, in particular the DRG neurons (dorsal root ganglion), and the sensory nerve fibres C-LTMRs (C-low-threshold mechanoreceptors), the latter being involved in the transmission of sensory signals (Delfini et al., 2013; Hoeffel et al., 2021; Salio et al., 2021).

Studies have shown that KO mice for TAFA4 are viable, without apparent abnormalities and respond to acute thermal and mechanical stimuli (Delfini et al., 2013). However, they have skin regeneration defects after exposure to UV (Hoeffel et al., 2021) and hypersensitivity to pain (Delfini et al., 2013, Kambrun et al., 2018; Salio et al., 2021). In particular, KO mice for TAFA4 have a 35-day post-irradiation inflammation resolution defect in an actinic-like erythema model via UVC irradiation. This condition can be compensated by intradermal injection of the post-irradiation recombinant TAFA4 protein (Hoeffel et al., 2021).

Moreover, KO mice for TAFA4 develop a significantly increased hypersensitivity to pain in pathological conditions (via intradural injection of inflammatory pain agonist or via partial lesion of the sciatic nerve) with respect to control mice. An intradural injection of recombinant human TAFA4 compensates for the condition of TAFA4 KO mice. In mice, TAFA4 also seems to play a restorative role during healing, in particular following a sunburn (Hoeffel et al., 2021). Furthermore, it could control allergic phenomena by inducing in particular the synthesis of IL-10, which in turn acts on inflammation of immune origin (Qiu et al., 2022).

In vivo on mice, an injection of the human TAFA4 protein, subcutaneously or at the level of the neurons present in the bone marrow, reduces the pain/hypersensitivity felt locally in the skin, up to 4 hours post-injection, in particular in a neuropathic pain model or a post-operative pain model (Kambrun et al., 2018; Yoo et al., 2021).

The use of the TAFA4 protein, peptide fragments composing it or a nucleic acid encoding it as an anti-inflammatory and soothing agent has already been proposed. Thus, document WO2014/180853A1 discloses the use of TAFA4 in pharmaceutical compositions making it possible to prevent, relieve or treat pain. Document WO2020/064907A1 describes the use of TAFA4 or a nucleic acid encoding it to combat skin inflammation. Document WO2021/156310A1 discloses the use of TAFA4 or a nucleic acid molecule encoding it to treat inflammatory diseases.

Although TAFA4 is described as an anti-inflammatory and soothing agent, its formulation in cosmetic or dermatological compositions, in particular for topical use, is difficult due in particular to issues of bioavailability, its molar mass of 15-20 kDa preventing its skin penetration, but also of maintaining biological activity, metabolisation by skin cells or microbiota bacteria, etc.

There is therefore a clear need to identify compounds, preferably non-pharmaceutical cosmetic compounds, in particular capable of being presented in a galenic form adapted to a topical application, advantageously ecobiological, which enable the prevention and/or care of sensitive and/or sensitised skin, in particular by increasing the skin tolerance threshold and/or by reducing skin discomfort, also by acting on the endogenous mechanisms of control of pain and inflammation specific to human skin.

### DETAILED DESCRIPTION OF THE INVENTION

Surprisingly, the Applicant, continuing their ecobiological approach in the search for action on the real causes of skin problems and to give the skin the means to strengthen itself, has demonstrated that the modulation of the endogenous content of TAFA4 and/or nucleic acid encoding it (i.e. modulation of the transcription and/or protein expression of TAFA4) was possible, in particular at the cutaneous level, more specifically by a topical intervention (excluding exogenous supply of TAFA4 and/or nucleic acid encoding it).

Even more surprisingly, the Applicant has demonstrated that the main living cells of the skin, namely keratinocytes and fibroblasts, also express TAFA4 in an endogenous manner, and that they can modulate this expression.

These major discoveries pave the way for a completely new management of skin sensitivity, in particular topically, in particular, cosmetically, which is more effective, more sustainable, and more respectful of the skin ecosystem, by acting respecting and via its own natural mechanisms thus explained.

The Applicant has thus identified that the modulation of the endogenous content of TAFA4 and/or of nucleic acid encoding it is an indicator making it possible to classify candidate compounds according to their capacity or not to prevent and/or treat sensitive and/or sensitised skin, in particular by increasing the skin tolerance threshold, in particular via the use of relevant experimental models.

Based on this observation, the Applicant has developed a reliable means (i.e. having an acceptable sensitivity and/or specificity), which is easy to read/interpret and can be integrated into a research and development routine, in particular in the cosmetic field, in order to decide on the selection or not of a compound, preferably ecobiological, intended for the prevention and/or care of sensitive and/or sensitised skin, this means being based on the modulation of the content of TAFA4 and/or nucleic acid encoding it.

Thus, a first aim of the present invention relates to a method for selecting at least one compound, preferably ecobiological, intended for the prevention and/or care of sensitive and/or sensitised skin, advantageously following a stress, more advantageously an allergy, implementing the identification of a modulation, advantageously of an increase, of the content of TAFA4 and/or nucleic acid, advantageously of mRNA, encoding it (i.e. encoding TAFA4). In other words, this is a method comprising, in particular, a step of identifying a modulation, advantageously an increase, of the content of TAFA4 and/or of nucleic acid, advantageously of mRNA, encoding TAFA4.

In particular, the method according to the invention comprises the following steps:
a) Bringing said at least one compound into contact with at least one skin cell;
b) Measuring the content of TAFA4 and/or nucleic acid, advantageously messenger ribonucleic acid (mRNA), encoding TAFA4;
c) Comparing the value obtained in step b) with a reference value;
d) Selecting said at least one compound inducing an increase in the content of TAFA4 and/or nucleic acid encoding TAFA4 in step c).

In the scope of the invention, the term “compound” means a substance or a compound which has biological and/or therapeutic properties which underlie a physiological and/or galenic effect. This can be an active principle, an excipient or mixtures thereof.

In the context of the invention, the terms “ecobiological composition”, “ecobiological compound”, “ecobiological ingredient”, “ecobiological active agent” or “ecobiological excipient” respectively mean a composition, a compound, an ingredient or an excipient which respects the person, their interactions with the world, and the planet. In particular, they mean a composition, a compound, an ingredient, an active agent or an excipient which respects the communities of living cells which constitute the skin (i.e. skin microbiota, keratinocytes, fibroblasts, etc.) which constantly interact with each other and with their environment.

In the scope of the invention, “ecobiological approach” means the particular approach initiated and developed by the inventor/Applicant which combines skin biology and ecology to help the skin live according to its natural mechanisms, over the long term.

In the scope of the present invention, the term “TAFA4” means, together and individually, any protein, polypeptide or peptide coming from a mammalian cell, advantageously human or murine, more advantageously human, corresponding to the active peptide sequence of the protein, including the different existing or predicted isoforms generated by alternative mRNAs, as well as all the peptide fragments composing it and all its biologically active derivatives. In particular, the amino acid sequence of TAFA4 corresponds to the sequence SEQ ID NO: 1 and/or to the sequence SEQ ID NO: 2. More specifically, “derivative” or “fragment” means a protein sequence identical to the sequence SEQ ID NO: 1 and/or to the sequence SEQ ID NO: 2 to at least 60%, preferably at least 70%, more preferably at least 80%, more preferably at least 85%, or even at least 90%, which therefore includes TAFA4 of different origins (non-human mammals, etc.).

In the scope of the invention, the term “biologically active derivatives” means isoforms, fragments or modified versions of isoforms or fragments, in particular covalently modified with grafts of organic groups, such as capryloyl, stearoyl, palmitoyl fatty chains or by acetylation, methylation, etc. Said derivatives can have a biological activity similar to or even greater than the protein from which they derive.

In the scope of the present invention, the term “nucleic acid encoding TAFA4” means, together and individually, any nucleic acid, preferably any ribonucleic acid, more preferably any messenger ribonucleic acid (mRNA), derived from a mammalian cell encoding for TAFA4, including its different isoforms. In particular, the nucleotide sequence of TAFA4 corresponds, partially or totally, to the sequence SEQ ID NO: 3, SEQ ID NO: 4, SEQ ID NO: 5, SEQ ID NO: 6 or SEQ ID NO: 7 or to a sequence identical to one of the sequences SEQ ID NO: 3 to 7 to at least 60%, preferably at least 70%, more preferably at least 80%, more preferably at least 85%, or even at least 90%, which therefore includes the nucleotide sequence encoding for a TAFA4 protein of different origins (non-human mammals, etc.). These complementary deoxyribonucleic acids (cDNAs) corresponding to as many alternative mRNAs identified in humans.

In the scope of the invention, the term “care” means a topical care, in particular cosmetic and non-therapeutic.

In the scope of the invention, the term “sensitised skin” means injured and/or attacked skin and/or in an inflammatory state and/or made painful and/or disordered, advantageously as a result of stress, more advantageously of an allergy.

In the scope of the invention, the term “brought into contact with at least one skin cell” means any direct (e.g. cells in culture) or indirect (e.g. by topical application) exposure to said at least one candidate compound according to the invention. In particular, indirect exposure by topical application can be carried out on the skin of a whole living organism, advantageously a human being, on a skin explant, on a reconstructed epidermis or on a reconstructed skin.

In the scope of the present invention, the term “skin explant” means a biopsy of human skin coming from surgical waste and which comprises the entire epidermis and a dermal and/or hypodermal compartment.

In the scope of the present invention, the term “skin substitute” means reconstructed skin or reconstructed epidermis, preferably human, which contains differentiated cells distributed in several layers. In particular, in the sense of the invention, the reconstructed skin contains at least two compartments, namely: a dermal compartment and an epidermal compartment. These models can be generated by 3D printing or by cell differentiation and can include several cell types in order to get as close as possible to the complete functionalities of the human or animal epidermis (innervation, immunisation, pigmentation, vascularisation, etc.). Examples of adapted models of this type are commercialised by the companies LabSkin Creations or Straticell, SkinEthic, Episkin or Phenion.

In the scope of the present invention, the term “reconstructed epidermis” means an epidermis generated in vitro by conventional techniques well known to a person skilled in the art (e.g. Limat and Hunziker 2002; Poumay et al., 2004). The keratinocytes used in this epidermis advantageously come from sensitive human skin.

In the scope of the present invention, the term “reconstructed skin” means an epidermal component, containing keratinocytes, generated in vitro and a dermal component, containing fibroblasts, generated in vitro integrated into a dermal matrix tissue. The reconstructed skin is commercially available. The keratinocytes and fibroblasts used in this skin can advantageously come from sensitive human skin.

In the scope of the present invention, the term “skin cells” means any cell type able to be found, at least partially, in one of the three layers of the skin, i.e. the cells of the epidermis (for example, keratinocytes, melanocytes, sebocytes, Merkel cells, immune cells such as Langerhans cells, nerve endings of type C fibres), cells of the dermis (for example, mast cells, vascular smooth muscle cells, specialised muscle cells, fibroblasts and immune cells, dermal dendritic cells, fibrocytes, endothelial cells), cells of the hypodermis (for example, adipocytes), nerve cells and macrophages. It is clear that according to the invention, the term “skin cell” comprises any neuronal cell, the ends or fibres of which are found in the skin. In particular, the skin cells are fibroblasts or keratinocytes, advantageously human, in particular, the skin cells are macrophages, advantageously M1-type macrophages, preferably human. The methods for obtaining M1 macrophage-like cells are known to a person skilled in the art, for example by differentiating THP-1 monocytes (Horiba et al., 2022) or PMBC monocytes (mononuclear cells of human peripheral blood; Wang et al., 2015). In particular, these are GINIP+ type, type C neurons (Salio et al., 2021; Jung et al., 2023).

In the scope of the invention, the terms “reference value”, “normal value”, “normal”, “standard”, “usual value” or “reference biological sample” are used interchangeably and mean a value or an average of the values of a parameter (e.g. gene expression, in particular at the mRNA level, etc.), coming from one or more subjects and representing the basal state of the considered measurement; as opposed to a “tested value”, “test value” or “test biological sample”. The reference value and the test value are obtained by implementing the same detection, quantification, identification method, etc.

According to a particular embodiment, the reference value according to the invention is the measurement obtained without bringing the at least one skin cell according to the invention into contact with the at least one compound according to the invention. In other words, the reference value according to the invention is the measurement obtained according to the same conditions (in particular, experimental, measurement, etc.) as in step b) but in the absence of step a) in the method according to the invention.

Preferably, the measurement of the reference value according to the invention is taken before or at the same time as step b) according to the invention, more advantageously under the same conditions, even more advantageously by using the same culture of the at least one skin cell.

In the scope of the invention, the terms “content”, “quantity” and “level” are used interchangeably.

In the scope of the invention, the term “content of TAFA4 and/or nucleic acid(s) encoding it” means the levels present in the cells (i.e. intracellular medium) and/or in the extracellular medium after secretion by said cells.

In the scope of the invention, the term “cutaneous content of TAFA4 and/or nucleic acid(s) encoding it” means the levels present in the skin cells (i.e. intracellular medium) and/or in the extracellular medium after secretion by said skin cells.

In the scope of the invention, the term “endogenous cutaneous content of TAFA4 and/or nucleic acid(s) encoding it” means the basal levels present in the skin cells (i.e. intracellular medium) and/or in the extracellular medium after secretion by said skin cells under normal conditions, in particular under normal culture conditions, and without induction nor depletion of the TAFA4 expression.

According to a particular embodiment, the content of TAFA4 and/or nucleic acid encoding TAFA4 according to the invention is measured in the intra- and/or extracellular medium of the at least one skin cell according to the invention.

In the scope of the invention, the term “intracellular medium” or “intracellular space” means the environment inside a cell where intracellular biological processes take place. It comprises the cytoplasm, the nucleus and the organelles. The term “extracellular medium” or “extracellular space” means the space outside a cell, composed of fluids such as blood, lymph, interstitial fluid and in particular, in the case of skin cells, the extracellular matrix or ECM, which is secreted by the cells of the skin. By definition, the secretion of intracellular constituents, like extracellular matrix precursors or cytokines, takes place in the extracellular medium.

In the scope of the invention, the term “modulation of the content of TAFA4 and/or TAFA4-encoding nucleic acid” means any modification of the content of TAFA4 and/or TAFA4-encoding nucleic acid, transcription and/or TAFA4 protein expression.

According to a particular embodiment, said modulation according to the invention corresponds to an increase in the content of TAFA4 and/or nucleic acid encoding it with respect to the reference value according to the invention. Preferably, the increase in the content of TAFA4 is at least 120%, more preferably 150%, even more preferably 200% with respect to the reference value according to the invention; and/or the increase in the content of nucleic acids encoding TAFA4 is at least 120%, more preferably 150%, even more preferably 200%, or even more than 1000% with respect to the reference value according to the invention.

In the scope of the invention, the term “variation of expression” means an overexpression or an underexpression of the gene. In particular, the term “overexpression” means that the expression of a gene is increased with respect to a reference value; the term “underexpression” means that the expression of a gene is decreased with respect to a reference value.

Unless otherwise indicated, in the sense of the invention, a “nucleotide sequence encoding for an amino acid sequence” means all nucleotide sequences which encode for the amino acid sequence, including degenerate nucleotide sequences making it possible to obtain said amino acid sequence. The nucleotide sequence which encodes a protein or RNA or cDNA can optionally comprise introns.

The terms “encoding” or “encoding for”, “encode” or “encode for” are used interchangeably and refer to the property inherent to the specific sequences of nucleotides in a polynucleotide, such as a gene, cDNA or mRNA, to serve as a matrix for the synthesis of polypeptides having a defined amino acid sequence, and the biological properties which result from them. Thus, a gene encodes for a protein if the transcription and translation of the mRNA corresponding to this gene produces the protein in a cell or another biological system. Both the encoding strand, the nucleotide sequence of which is identical to the mRNA sequence and which is generally described in sequence listings and databases, and the non-encoding strand, used as a matrix for the transcription of a gene or cDNA, can be designated as encoding for the protein or another product of this gene or cDNA.

In the sense of the invention, the terms “peptide”, “polypeptide” and “protein” are used interchangeably and make reference to a compound consisting of amino acid residues covalently linked by peptide bonds. A protein by definition contains at least two amino acids, without limitation as regards the maximum number of amino acids. The polypeptides equally comprise several peptides and/or proteins, which themselves comprise two or more amino acids linked to one another by peptide bonds. Such as used in this case, the term refers to both short chains, which are also commonly designated in the art as peptides, oligopeptides and oligomers for example, and longer chains, which are generally designated in the art as proteins, of which there are many types. The “polypeptides” comprise, for example, biologically active fragments, substantially homologous polypeptides, oligopeptides, homodimers, heterodimers, polypeptide variants, modified polypeptides, derivatives, analogues, fusion proteins, among others. The polypeptides comprise natural peptides, recombinant peptides, synthetic peptides, or a combination thereof.

The terms “identical” and “similar” refer to sequence similarity, which is measured as a percentage of sequence identity between two polypeptides or between two nucleic acid molecules. When a position in each of the two compared sequences is occupied by the same amino acid monomer base or subunit (for example, when a position in each of the two DNA molecules is occupied by an adenine), then the molecules are identical for this position. The percentage of identity, or similarity between two sequences is according to the number of corresponding positions shared by the two sequences, and corresponds to this number divided by the number of positions compared and multiplied by 100. For example, if 6 out of 10 positions in two paired sequences are identical, then both sequences are 60% identical or similar. As a general rule, the comparison is made by aligning the two sequences so as to give maximum identity and, concerning the protein sequences, without taking into account preservative substitutions as forming part of the sequence identity.

In the scope of the invention, the term “amplification primer” means a nucleotide fragment being able to comprise from 5 to 100 nucleotides, preferably from 15 to 30 nucleotides, and having a specificity of hybridisation with a target nucleotide sequence, under conditions determined for the initiation of an enzymatic polymerisation, for example, in an enzymatic amplification reaction of the target nucleotide sequence. Generally, “primer pairs” are used, consisting of two primers which hybridise to the two complementary strands. When it is sought to perform the amplification of several different genes, several pairs of different primers are preferably used, each preferably having an capacity to hybridise specifically with a different gene.

In the scope of the invention, the term “hybridisation probe” means a nucleotide fragment typically comprising from 5 to 100 nucleotides, preferably from 15 to 90 nucleotides, even more preferably from 15 to 35 nucleotides, having a hybridisation specificity under conditions determined to form a hybridisation complex with a target nucleotide sequence. The probe also comprises a reporter (such as a fluorophore, an enzyme or any other detection system), which will enable the detection of the target nucleotide sequence. In the present invention, the target nucleotide sequence can be a nucleotide sequence comprised in a messenger RNA (mRNA) or a nucleotide sequence comprised in a complementary DNA (cDNA) obtained by reverse transcription of said mRNA. When it is sought to target several different genes, several different probes are preferably used, each preferably having a capacity to specifically hybridise with a different gene.

In the scope of the invention, “hybridisation” means the process during which, under suitable conditions, two nucleotide fragments, such as for example, a hybridisation probe and a target nucleotide fragment, having sufficiently complementary sequences, are able to form a double strand with stable and specific hydrogen bonds. A nucleotide fragment “capable of hybridising” with a polynucleotide is a fragment being able to hybridise with said polynucleotide under hybridisation conditions, which can be determined in each case in a known manner. The hybridisation conditions are determined by the precision of the operating conditions. Hybridisation is all the more specific that it is carried out with greater precision. This is defined, in particular, according to the base composition of a probe/target duplex, as well as by the degree of mismatch between two nucleic acids. All these data are well-known and the suitable conditions can be determined by a person skilled in the art. In general, according to the length of the hybridisation probes used, the temperature for the hybridisation reaction is between around 20 and 70° C., in particular between 35 and 65° C. in a saline solution at a concentration of about 0.5 to 1M. A step of detecting the hybridisation reaction is then carried out.

In the scope of the invention, the term “enzymatic amplification reaction” means a process generating multiple copies of a target nucleotide fragment, by the action of at least one enzyme. Such amplification reactions are well-known to a person skilled in the art and the following techniques, in particular, can be mentioned: Polymerase Chain Reaction (PCR), Ligase Chain Reaction (LCR), Repair Chain Reaction (RCR), Self Sustained Sequence Replication (3SR) with document WO-A-90/06995, Nucleic Acid Sequence-Based Amplification (NASBA), Transcription Mediated Amplification (TMA) with document U.S. Pat. No. 5,399,491 and Loop mediated isothermal amplification (LAMP) with document U.S. Pat. No. 6,410,278. When the enzymatic amplification reaction is a PCR, more specifically, RT-PCR (RT for “reverse transcription”) will be preferably referred to, when the amplification step is preceded by a step of reverse transcription of messenger RNA (mRNA) into complementary DNA (cDNA), and of qPCR or RT-qPCR when the PCR is quantitative.

In the scope of the invention, the term “inducer” and the term “inducer of the synthesis of TAFA4 and/or nucleic acid encoding it” are used interchangeably and mean an agent, a treatment or a culture condition capable of triggering a determined reaction or a programmed sequence of reactions in the biological system, namely the production (i.e. transcription and/or translation) of TAFA4 and/or of nucleic acid encoding it.

In the scope of the invention, the term “inhibitor” and the term “inhibitor of the synthesis of TAFA4 and/or nucleic acid encoding it” are used interchangeably and mean an agent, a treatment or a culture condition capable of triggering a determined reaction or a programmed sequence of reactions in the biological system, namely the partial or total inhibition of the production (i.e. transcription and/or translation) of TAFA4 and/or of nucleic acid encoding it.

In the scope of the invention, the term “depletion of the endogenous cutaneous content of TAFA4 and/or nucleic acid encoding it” means the application of an agent, a treatment or a culture condition capable of inducing a decrease or even a removal of the content of TAFA4 and/or of nucleic acid encoding it, in the biological system.

In the scope of the invention, the terms “sensitising agent” or “irritating agent” or “polluting agent” are used interchangeably and mean an agent, a treatment or a culture condition causing skin dysregulation or sensitisation, namely skin having different physiological constants to degrees which deviate from normal, in particular having an inflammatory state such as that observed during an increase in the expression and/or concentration of IL-8, IL-1, TNFα or other inflammatory cytokines. Advantageously, such an agent can be a surfactant, more advantageously, sodium lauryl sulfate (SDS), or also a preservative, more advantageously methylisothiazolinone, or another substance, not corresponding to a cosmetic raw material, but which can be found as a contaminant of cosmetic raw materials, more advantageously a chromium salt, a nickel salt, cobalt, or a substance present in the environment, more advantageously microparticles, pollen.

In the scope of the invention, the term “non-sensitised cell” means a cell coming from a donor which is not characterised by a sensitive and/or sensitised skin, and which has not been subjected to a stress which can sensitise it.

Preferably, the present invention aims for a method for selecting at least one compound, such as defined above and having the following technical features, taken individually or in combinations:
Said modulation of the content of TAFA4 and/or nucleic acid encoding TAFA4 is an increase;
Said increase in the content of TAFA4 corresponds to at least 120%, more preferably 150%, even more preferably 200% with respect to the reference value according to the invention;
Said increase in the content of nucleic acid encoding TAFA4 corresponds to at least 120%, more preferably 150%, even more preferably 200%, or even more than 1000% with respect to the reference value according to the invention;
the at least one compound is not the TAFA4 protein and/or one of its biologically active derivatives and/or one of its isoforms;
said method is an in vivo, in vitro or ex vivo method;
said method is an in vivo or ex vivo method and is characterised in that the bringing into contact of step a) is performed by topical application of said at least one compound to the skin of a whole living organism, advantageously the skin of a whole non-human living organism, a skin explant, a reconstructed epidermis or a reconstructed skin;
said method is an in vivo method in a human subject;
said method is an in vivo method in a non-human subject, advantageously a rodent (e.g. mouse, rat, guinea pig or rabbit), a cat, a dog, a primate (e.g. a chimpanzee), a bird (e.g. a chicken), a reptile, an amphibian or a fish (e.g. zebrafish);
said method is an in vitro method comprising the culture of skin cells, advantageously selected from the group consisting of keratinocytes, melanocytes, sebocytes, Merkel cells, vascular smooth muscle cells, specialised muscle cells, fibroblasts, mast cells, lymphocytes, monocytes, dendritic cells, advantageously dermal dendritic cells; Langerhans cells, adipocytes, nerve cells, macrophages, advantageously M1 or M2 type macrophages; endothelial cells; and mixtures thereof; preferably chosen from among keratinocytes, fibroblasts and mixtures thereof; and is characterised in that the bringing into contact of step a) is carried out by adding into the culture medium;
said at least one skin cell is at least one macrophage, advantageously at least one M1-type macrophage, preferably at least one human M1-type macrophage;
said at least one skin cell is at least one keratinocyte, advantageously at least one human keratinocyte, more advantageously at least one normal human keratinocyte;
said at least one skin cell is at least one fibroblast, advantageously at least one human fibroblast, more advantageously at least one normal human fibroblast;
said method further comprises, before the implementation of step a), a step of adding into the culture medium, an inducer of the synthesis of TAFA4 and/or nucleic acid encoding it, by said at least one skin cell, advantageously chosen from among: phorbol 12-myristate 13-acetate (Pam), preferably at a concentration of between 1 and 20 ng/ml; lipopolysaccharide (LPS), preferably at a concentration of between 0.1 μg/ml and 2 mg/ml; calcium, preferably at a concentration of between 0.1 mM and 10 mM; forskolin, preferably at a concentration of between 10 μM and 200 μM; the combination of calcium, preferably at a concentration of between 0.1 mM and 10 mM, and forskolin, preferably at a concentration of between 10 μM and 200 μM; epidermal growth factor (EGF), preferably at a concentration of between 10 ng/ml and 200 ng/ml; faetal bovine serum (FBS), advantageously in a concentration of between 0.1 and 5% by weight of the total culture medium; exposure to UV light, preferably exposure to a dose of between 2 and 10 J/cm² of UVA for at least 5 minutes, preferably at least 10 minutes; and mixtures thereof;
said addition step is carried out in an interval of between 30 minutes and 72 hours, preferably under the following culture conditions: 37° C., 5% CO₂ and/or atmosphere saturated with humidity;
said method further comprises, before implementing step a), a step of adding into the culture medium, an inhibitor of the synthesis of TAFA4 and/or nucleic acid encoding it, by said at least one skin cell;
said method further comprises, before implementing step a), a step of depleting the endogenous cutaneous content of TAFA4 and/or nucleic acid encoding it produced by said at least one skin cell;
said step b) of measuring the content of TAFA4 and/or nucleic acid, advantageously mRNA, encoding it is carried out at a protein level by an ELISA test, immunohistochemistry (including colorimetry, fluorescence, luminescence), other colorimetric method, or western blot, dot blot, western simplified by capillary immunoelectrophoresis (WES) and/or at the nucleic level by in situ hybridisation, northern blot, sequencing or RT-PCR, advantageously RT-qPCR;
said content of TAFA4 and/or nucleic acid, advantageously mRNA, encoding TAFA4 is measured in the intra- and/or extracellular medium of said at least one skin cell;
before implementing said step a), said at least one skin cell has been brought into contact, preferably by topical application or by adding into the culture medium, with a sensitising agent, advantageously a cosmetic sensitising agent, more advantageously, a surfactant or a preservative;
the amino acid sequence of TAFA4 corresponds, partially or totally, to the sequence SEQ ID NO: 1 and/or to SEQ ID NO: 2, or to a sequence identical to one of the sequences SEQ ID NO: 1-2 to at least 60%, preferably at least 70%, more preferably at least 80%, more preferably at least 85%, or even at least 90%;
the nucleotide sequence of TAFA4 corresponds, partially or totally, to the sequence SEQ ID NO: 3, SEQ ID NO: 4, SEQ ID NO: 5, SEQ ID NO: 6 or SEQ ID NO: 7, or to a sequence identical to one of the sequences SEQ ID NO: 3-7 to at least 60%, preferably at least 70%, more preferably at least 80%, more preferably at least 85%, or even at least 90%;
said method comprises, in said step b), the additional measurement of the content of at least one indicator agent different from TAFA4 and/or the nucleic acid encoding it, for example IL-8, IL-1, TNF-α or other anti-inflammatory cytokines, said content of indicator agent also being compared with a reference value specific to said step c);
said reference value is the measurement obtained without bringing the at least one skin cell into contact with said at least one compound;
said reference value is the measurement obtained under the same conditions as in said step b) but in the absence of said step a);
the measurement of said reference value is taken before or at the same time as said step b) according to the invention, more advantageously under the same conditions, even more advantageously using the same culture of said at least one skin cell; —said selection step d) is associated with another selection step based on the induction of an increase or a decrease in the content of said indicator agent (modulation dependent on the chosen indicator agent) to decide on the selection or not of said at least one compound;
said method further comprises, after implementing step d), a step e) of formulating said at least one compound selected from a cosmetic and/or food composition, non-therapeutic, advantageously ecobiological, more advantageously being presented in a galenic form suitable for topical and/or oral administration;
said step e) of formulating said at least one selected compound corresponds to the addition of at least one active ingredient usually used in the cosmetic, food and/or dermatological fields, advantageously selected from an anti-radical agent or more generally an antioxidant, a whitening agent, a pigmenting agent, an emollient, a moisturiser, anti-seborrhoeic agents, an anti-inflammatory agent, an anti-acne agent, a keratolytic and/or desquamating agent, an anti-wrinkle and/or tightening agent, a mineral or organic sunscreen which is hydrophilic or lipophilic, a draining agent, an anti-irritant agent, a soothing agent, a vitamin and mixtures thereof, a mattifying agent, an anti-aging active agent such as retinol, a healing agent, an antiseptic and an essential oil; and
said step e) of formulating said at least one selected compound corresponds to the addition of at least one excipient usually used in the cosmetic, food and/or dermatological fields, advantageously selected from among a fat, an emulsifier, a co-emulsifier, a hydrophilic or lipophilic gelling agent, a preservative, an antioxidant, a solvent, an exfoliating agent, a perfume, a filler, a neutralising agent, a pro-penetrating agent, and a polymer.

Thus, and according to an aspect of the invention, the method according to the invention is an in vitro method, said bringing into contact performed by adding to the culture medium, and the at least one skin cell is chosen from among keratinocytes, melanocytes, sebocytes, Merkel cells, vascular smooth muscle cells, specialised muscle cells, fibroblasts, mast cells, lymphocytes, monocytes, dendritic cells, advantageously dermal dendritic cells; Langerhans cells, adipocytes, nerve cells, macrophages, advantageously of type M1 or M2; endothelial cells; and mixtures thereof.

According to one aspect, the at least one skin cell is chosen from among keratinocytes and fibroblasts, preferably human, more preferably normal; and mixtures thereof.

According to an aspect of the invention, the at least one skin cell is a murine cell, advantageously a cell coming from an immortalised murine line of B16 melanocytes.

The measurement of the content of TAFA4 and/or nucleic acid, advantageously of mRNA, encoding it can be performed with any method known to a person skilled in the art. As an example, Northern blot, Southern blot, PCR, RT-PCR, quantitative RT-PCR, SAGE and its derivatives, nucleic acid chips, including cDNA chips, oligonucleotide chips and mRNA chips, tissue chips and RNA-Seq, immunohistology methods, immunoprecipitation, western blot, western simplified by capillary immunoelectrophoresis (WES), dot blot, ELISA or ELISPOT, protein chips, antibody chips, or tissue chips coupled to immunohistochemistry, FRET or BRET techniques, microscopy or histochemistry methods, including in particular confocal microscopy and electron microscopy methods, methods based on the use of one or more excitation wavelengths and a suitable optical method, such as an electrochemical method (voltammetry and amperometry techniques), the atomic force microscope, and radiofrequency methods, such as multipolar, confocal and non-confocal resonance spectroscopy, fluorescence detection, luminescence, chemiluminescence, absorbance, reflectance, transmittance, and birefringence or refractive index (e.g. surface plasmon resonance, ellipsometry, resonant mirror method, etc.), flow cytometry, isotopic or magnetic radio resonance imaging, polyacrylamide gel analysis; electrophoresis (SDS-PAGE) HPLC spectrophotometry, liquid chromatography/mass spectrophotometry/mass spectrometry (LC-MS/MS) can be mentioned.

Advantageously, the quantification of the content of TAFA4 can be done by immunohistochemistry or immunocytochemistry on sections of skin or cells in culture and other methods; by western blot; by dot blot; by western simplified by capillary immunoelectrophoresis (WES). In particular, the protein dot blot is based on the principle of hybridisation between a probe, generally labelled antibodies, and the sought target. It is simple and rapid, not requiring any complex purification steps. To make a dot blot, the biological sample (cell supernatants or protein extracts) is deposited on a nitrocellulose or nylon membrane, thus forming small dots. After drying, the membrane is incubated with the probe, in order to detect the sought protein. The Western method simplified by capillary immunoelectrophoresis (WES) combines the principles of the Western blot technique and capillary immunoelectrophoresis to detect and quantify specific proteins in a biological sample without going through membrane transfer, a laborious procedure. In this approach, the proteins are first separated by capillary electrophoresis according to their size and their electrical charge; the detection of the target proteins is then carried out by using specific antibodies labelled directly on the capillaries, followed by an automated analysis in a dedicated apparatus. Alternatively, the quantification of the content of TAFA4 can be done by an ELISA test, for example, on cell culture supernatants or cell extracts, by interpolating the optical density of the sample on a calibration curve obtained with serial dilutions of the recombinant human TAFA4 protein (for example, from 0.1561 ng/ml to 10 ng/ml). In practice, on the cell layer, a viability test with ((3-4,5-dimethylthiazol-2-yl)-2,5-diphenyl tetrazolium bromide) or MTT, known to a person skilled in the art, can be carried out. TAFA4 assays are used, only if cell viability is greater than 80%, and are related to this cell viability value.

The measurement of the content of messenger ribonucleic acid (mRNA) encoding TAFA4 can advantageously be taken by in situ hybridisation on cell sections; by northern blotting; or also by RT-qPCR. Advantageously, total RNA is extracted from tissues, for example using commercially available kits (like Rneasy Plus Mini QIAGEN). The extraction can be followed by an assay of the total RNAs, which makes it possible to know the quantity and quality of the RNAs in the cell extract; this step can be carried out, for example, using the Bioanalyser and the RNA 6000 Nano kit. The cDNA is generated using the QuantiTect Reverse Transcription kit (QIAGEN), followed by real-time Quantitative PCR at each treatment time for the TAFA4 gene, using, for example, the QuantiFast SYBR Green PCR Kit (QIAGEN), and following the expression of a housekeeping gene (for example, GAPDH), as a control and TAFA4 with specific primers based on SEQ ID NOs: 3, 4, 5, 6 or 7.

Thus, and according to a particular embodiment, the measurement of the content of TAFA4 is carried out by an ELISA test, by immunohistochemistry on cell sections; by western blotting; the measurement of the content of messenger ribonucleic acids (mRNA) encoding TAFA4 is carried out by immunohistochemistry, in situ hybridisation on cell sections, by northern blotting, sequencing or RT-PCR, advantageously RT-qPCR.

The quantification of the content of at least one marker different from TAFA4 such as mentioned above, and/or of the nucleic acid encoding it can be done according to the methods specified above.

According to a particular embodiment, the method for selecting at least one compound, preferably ecobiological, intended for the prevention and/or care of sensitive and/or sensitised skin, advantageously following a stress, more advantageously an allergy, according to the invention comprises the following steps:
a) Bringing said at least one compound into contact in vitro with at least one skin cell by adding it into the culture medium;
b) Measuring the content of TAFA4 and/or nucleic acid encoding TAFA4, preferably in the culture medium and/or in the intracellular medium of said at least one skin cell;
c) Comparing the value obtained in step b) with a reference value, preferably obtained without bringing said at least one skin cell into contact with said at least one compound;
d) Selecting said at least one compound inducing an increase in the content of TAFA4 and/or nucleic acid encoding it in step c), preferably by more than 120%, advantageously by more than 150%, with respect to said reference value.

According to an alternative embodiment, the method for selecting at least one compound, preferably ecobiological, intended for the prevention and/or care of sensitive and/or sensitised skin, advantageously following a stress, more advantageously an allergy, according to the invention, comprises the following steps:
a) Bringing said at least one compound into contact ex vivo with at least one skin cell by topical application to a skin explant, a skin substitute, a reconstructed epidermis or a reconstructed skin;
b) Measuring the content of TAFA4 and/or nucleic acid encoding it, preferably in the intra- and/or extracellular medium of said at least one skin cell;
c) Comparing the value obtained in step b) with a reference value, preferably obtained without bringing said at least one skin cell into contact with said at least one compound;
d) Selecting said at least one compound inducing an increase in the content of TAFA4 and/or nucleic acid encoding it in step c), preferably by more than 120%, advantageously by more than 150%, with respect to said reference value.

According also to another embodiment, the method for selecting at least one compound, preferably ecobiological, intended for the prevention and/or care of sensitive and/or sensitised skin, advantageously following a stress, more advantageously an allergy, according to the invention comprises the following steps:
a) Bringing said at least one compound into contact in vivo with at least one skin cell by topical application to the skin of a whole living organism, advantageously a human or non-human being;
b) Measuring the content of TAFA4 and/or nucleic acid encoding it, preferably in the intra- and/or extracellular medium of said at least one skin cell;
c) Comparing the value obtained in step b) with a reference value, preferably obtained without bringing said at least one skin cell into contact with said at least one compound;
d) Selecting said at least one compound inducing an increase in the content of TAFA4 and/or nucleic acid encoding it in step c), preferably by more than 120%, advantageously by more than 150%, with respect to said reference value.

According to a particular embodiment, said at least one skin cell, to be used in the method according to the invention, is not sensitised.

According to an alternative embodiment, said at least one skin cell, to be used in the method according to the invention, is sensitised.

According to another embodiment, said at least one cell comes from a donor characterised by sensitive or sensitised, advantageously irritable, or reactive skin.

According to an alternative embodiment, said at least one cell has previously been brought into contact, by topical application or by adding into the culture medium, with a sensitising agent, advantageously a cosmetic sensitising agent, even more advantageously chosen from among a surfactant and a preservative.

According to a particular aspect of the invention, the endogenous cutaneous contents of TAFA4 and/or of the nucleic acid encoding it of said cell according to the invention have been depleted, before said cell was brought into contact with the at least one candidate compound.

According to a particular aspect, said compound according to the invention does not correspond to the TAFA4 protein nor to one of its isoforms or to one of its biologically active derivatives or to TAFA4 or to a ribonucleic acid encoding it or to a nucleic acid encoding it, or to their formulation.

Another aim of the present invention relates to a kit for the implementation of the method for selecting at least one compound, preferably ecobiological, intended for the prevention and/or care of sensitive and/or sensitised skin, advantageously following a stress, more advantageously an allergy, such as defined above, comprising means for amplification and/or means for detection of the content of TAFA4 and/or nucleic acid encoding it, advantageously said means for amplification and/or detection are specific reagents of the expression products of TAFA4 target genes chosen from among amplification primers, hybridisation probes or also antibodies which specifically bind to TAFA4, advantageously the TAFA4 protein and/or one of its biologically active derivatives.

Another aim of the present invention also relates to a composition comprising at least one compound selected by the method described above.

Preferably, the present invention aims for a composition such as described above and having the following technical features, taken individually or in combinations:
the composition comprises at least one compound selected by the method described above;
the composition consists of at least one compound selected by the method described above;
the composition is presented in at least one of the suitable galenic forms for a topical application on the skin and/or the mucous membranes, and/or the dander, for example in anhydrous form, in the form of an oil-in-water emulsion, of a water-in-oil emulsion, of a multiple emulsion, of a silicone emulsion, or a microemulsion, of a nanoemulsion, of a gel, of an aqueous solution or of a hydroalcoholic solution;
the composition is fluid or not, and is presented in the form of a cream, of an ointment, of a milk, of a lotion, of a serum, or of a gel;
the composition is presented in at least one of the galenic forms suitable for a non-therapeutic food application, advantageously orally; and
the composition is coloured or not, advantageously coloured by means of a colouring agent.

Another aim of the present invention also relates to the use of the modulation of the content of TAFA4 and/or nucleic acid, advantageously of mRNA, encoding it, as an indicator making it possible to classify candidate compounds according to their capacity or not to increase the skin tolerance threshold.

Another aim of the present invention also relates to a method for formulating a cosmetic and/or food composition, non-therapeutic, advantageously ecobiological, more advantageously being presented in a galenic form suitable for topical and/or oral administration, comprising the incorporation into said composition of at least one compound selected by the selection method according to the invention.

The examples below, without being limiting, form an integral part of the invention and any feature which appears to be novel with respect to the prior art is claimed as such and as a general means.

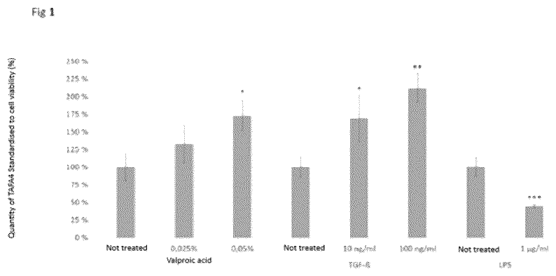

FIG. 1 shows the effect of valproic acid, TGF-β and LPS tested at different concentrations and LPS at a concentration (1 μg/ml) on the synthesis of TAFA4 in normal human fibroblasts (NHF) expressed as a percentage of induction. NS: p>0.05; *: p<0.05; **: p<0.01; ***: p<0.001.

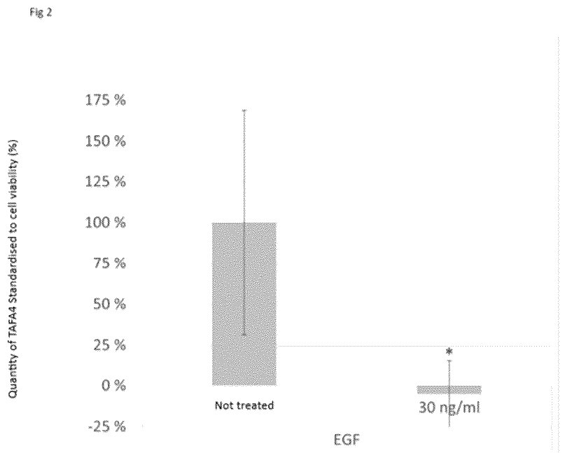

FIG. 2 shows the effect of EGF protein at a concentration (30 ng/ml) on the synthesis of TAFA4 in normal human keratinocytes (NHF) expressed as a percentage of induction. NS: p>0.05; *: p<0.05; **: p<0.01; ***: p<0.001.

### EXAMPLES OF EMBODIMENTS

### Example I—Keratinocyte and Fibroblast Protocol

### I.1 Introduction

The aim of the study is to provide a simple and rapid protocol making it possible to study the modulation of the TAFA4 expression or transcription in keratinocyte and fibroblast lines by cosmetic compounds and compositions. It must be noted that, optionally, an inducer of TAFA4 expression can be implemented to potentiate the expression of the gene encoding this protein, in particular in the case where the amount of gene encoding the TAFA4 protein or the amount of TAFA4 protein is less than or equal to the detection limit imposed by the method implemented by a person skilled in the art and/or to provide a reference TAFA4 production value, i.e. a positive control, this value having to be reached in order to classify the candidate compound in the category of compounds, preferably ecobiological, capable for the prevention and/or care of sensitive and/or sensitised skin.

### I.2 Cell Culture

Normal human keratinocytes (NHK) and normal human fibroblasts (NHF) have been used in this study. They come from Promocell (France), Lonza (France).

The conditions tested for the development of the model are:
Cell Type: NHK, NHF
Culture medium: KBM+1% P/S for NHK, DMEM+1% P/S for NHF.
Processing time: 48 hours and 72 hours
Seeding density: 15,000 cells/well in 96 well plates at time 48 hours

10,000 cells/well in 96-well plates at time 72 hours.

We have chosen to test different induction conditions for the 2 cell types:

These inducers have been selected as positive controls based on data from the bibliography.
24-hour pretreatment with PMA at 10 ng/ml (P1585-1 MG, SIGMA)
24-hour pretreatment with PMA at 50 ng/ml
1 mg/ml LPS (L2880-10 MG, SIGMA)
LPS at 1 mg/ml+PMA at 10 ng/ml (24-hour pretreatment for PMA)
LPS at 1 mg/ml+PMA at 50 ng/ml (24-hour pretreatment for PMA)
Calcium 1.2 mM (C7902-500G, SIGMA)
Calcium 1.2 mM+Forskolin at 10 μM
100 μM Forskolin (93049-10 MG, SIGMA)
10 μM Forskolin

Specifically for NHKs:
10 ng/ml EGF (AF100-15, Peprotech)
30 ng/ml EGF
10 ng/ml EGF+100 μM Forskolin
30 ng/ml EGF+100 μM Forskolin

Specifically for NHFs
0.5% FBS (S1900-500B, Dominique Dutscher)
2% FBS
0.5% FBS+100 μM Forskolin
2% FBS+100 μM Forskolin

### I.3 Screening of Compounds or Compositions

The cells (NHK, NHF) are seeded in a 96-well plate in the corresponding complete culture medium. After 24 hours of culture at 37° C., 5% CO₂, the compounds or compositions to be evaluated are added to the chosen concentrations in the culture medium and the cells are incubated for 48 hours or 72 hours at 37° C., 5% CO₂. A positive induction control is evaluated in parallel with the active agents.

After the different incubation times, the supernatants are collected and preserved at −80° C. before performing the TAFA4 assay by ELISA. The assay is performed only if the cell viability is greater than 80%.

For the evaluation of TAFA4 expression by RT-qPCR, the cells are recovered after incubation of the active agents or positive controls on shorter kinetics (30 minutes to 24 hours).

### I.4 Selection of Compounds or Compositions to be Evaluated

Different pharmaceutical or cosmetic-type molecules can be preselected and tested. These molecules will be directly solubilised in the culture medium or solubilised in ethanol or DMSO according to their nature.

### I.5 Assay of TAFA4 in Culture Supernatants by ELISA

The quantification of TAFA4 in supernatants or cell extracts is done thanks to an ELISA test, Enzyme Linked Immunosorbent Assay (Ref: abx522987, Cliniscience, France) with the reagents recommended by the manufacturer.

The Optical Density (OD) is read with a spectrophotometer at 450 nm. The concentration of TAFA4 is calculated from the standard range (0.1561 ng/ml to 10 ng/ml) established with the recombinant human TAFA4 protein. The results are expressed in ng/ml of TAFA4 per well. On the cell layer, an MTT viability test is performed. TAFA4 assays are used, only if cell viability is greater than 80%, and are related to this cell viability value.

### I.6 Quantification of the TAFA4 Expression by RT-qPCR

After 30 minutes to 24 hours of incubation with the active agents, an extraction of the total RNAs is carried out using the Rneasy Plus Mini kit according to the recommendations of the supplier (Qiagen).

The assay of total RNAs makes it possible to know the quantity and quality of RNAs in the cell extract. The assay is performed using the Bioanalyser and the RNA 6000 Nano kit.

Reverse Transcription performed using the QuantiTect Reverse Transcription kit (Qiagen) makes it possible to obtain cDNA.

A real-time Quantitative PCR at each treatment time is performed for the TAFA4 gene. The Qiagen QuantiFast SYBR Green PCR kit is used.
Housekeeping gene: GAPDH.
Gene of interest: TAFA4→see the specific primers of the gene

### I.7 MTT Cell Viability Test

After incubation and collection of the culture supernatants, the wells are rinsed with D-PBS. After removal of D-PBS, 100 μl of 1 mg/ml MTT solution are added per well and incubated for 3 hours at 37° C. The MTT is then removed and 100 μl of DMSO is added into each well to dissolve the formazan crystals. After homogenisation of the colouration, the optical density is read with a spectrophotometer at 540 nm (Victor 3, Perkin Elmer). The results are then expressed from the formula: % viability=(OD condition treated/OD (100% viability))×100. The cells which represent 100% viability correspond to the cells incubated at the different times in the control culture medium.

NB: MTT ((3-4,5-dimethylthiazol-2-yl)-2,5-diphenyl tetrazolium bromide) is a soluble yellow colourant which is metabolised by mitochondrial enzymes (succinate dehydrogenase) into a dark blue compound: formazan. The formazan crystals are then solubilised in DMSO and the optical density is measured spectrophotometrically at 540 nm.

### I.8 Statistical Analyses

The data have been collected from independent experiments performed in triplicate. Quantitative analyses are expressed in the form of mean±standard deviation. Statistical significance is determined by a Student test. The differences are considered as statistically significant from p<0.05.

(NS: p>0.05; *: p<0.05; **: p<0.01; ***: p<0.001).

### Example II—Macrophage Protocol

### II.1 Introduction

The purpose of the study is to provide a simple and rapid protocol to study the modulation of the TAFA4 expression or transcription in an inflammatory macrophage model by cosmetic compounds and compositions. It is noted that a depletion of the content of TAFA4 (depletion of the expression of the gene encoding the TAFA4 protein and/or the quantity of TAFA4 protein) can be implemented to provide a model in which the endogenous constitutive production of TAFA4 must be restored in order to classify the candidate compound in the category of compounds, preferably ecobiological, intended for the prevention and/or care of sensitive and/or sensitised skin.

### II.2 Cell Culture

Macrophages are immune cells capable of infiltrating tissues. The macrophages are differentiated from monocytes (blood cells), themselves differentiated from bone marrow stem cells. Macrophages and monocytes are cells capable of phagocytosis. Their role is to phagocyte cellular debris and pathogens and they contribute more broadly to the immune response by synthesising different types of mediators.

There are 3 main types of macrophages:
MOs which are non-activated macrophages.
M1s which have pro-inflammatory properties. At the cutaneous level, they are, in particular, involved in the destruction of pathogens, in particular, during healing.
M2s which have anti-inflammatory and pro-resolution properties of inflammation. At the cutaneous level, they are, in particular, involved in the repair phase of healing.

At the vitro level, there are different cell models approaching a macrophage-type phenotype.

These different models can be used in this scope:
Human monocyte lines: THP-1, U-937, SC, AML-193, HL-60/S4

THP-1s are the cells the most described and used in literature.

For THP-1s: Culture medium: RPMI 1640 (Roswell Park Memorial Institute)+10% FBS (faetal bovine serum) (Wang et al., 2015)+/−100 mg/L gentamycin, 4.5 g/L glucose, 1 mM pyruvate, 0.05 mM 2-mercaptoethanol and 2 mM L-glutamine (Shen et al., 2014) and +/−penicillin (100 units/mL) and streptomycin (100 μg/mL) in a humid environment with 5% CO₂ in the air and at 37° C. (Kawano et al., 2015).

THP-1s can be differentiated into MO, M1 and M2 macrophages according to the culture conditions described by Horiba et al., 2022.
Macrophages derived from monocytes coming from human PBMCs (human peripheral blood mononuclear cells) coming from human peripheral blood samples.

PMBCs are isolated from blood by Polymorphprep. The monocytes (CD14+) are then recovered after 4 hours of adhesion of the PMBCs. Culture medium: RPMI 1640+10% FBS. Monocytes can be differentiated into MO macrophages by M-CSF (macrophage colony-stimulating factor) stimulation in vitro and then by treatment with LPS (Escherichia coli 055: B5), combined with IFN-γ (gamma interferon) or IL-4 (interleukin-4), to differentiate them into M1 or M2 macrophages respectively, for 24 hours (Wang et al., 2015) in a humid environment with 5% CO₂ in the air and at 37° C.
Macrophages derived from CD34+ haematopoietic stem cells

These cells are recovered from samples from bone marrow, peripheral blood, and human umbilical cord blood in vivo (Clanchy and Hamilton, 2013). Conventionally, these cells are recovered from cord blood.

### II.3 Selection of Compounds or Compositions Effective to Induce the Expression or Synthesis of TAFA4

### II.3.1. Protocol

The cells are seeded in a 96-well plate in the corresponding complete culture medium. After 24 hours of culture at 37° C., 5% CO₂, the active agents to be evaluated are added to the chosen concentrations in the culture medium and the cells are incubated for 48 hours or 72 hours at 37° C., 5% CO₂. A positive induction control is evaluated in parallel with the active agents.

After the different incubation times, the supernatants are collected and preserved at −80° C. before performing the TAFA4 assay by ELISA. The assay is performed only if the cell viability is greater than 80%.

For the evaluation of the TAFA4 expression by RT-qPCR, the cells are recovered after incubation of the active agents or positive controls on shorter kinetics (30 minutes to 24 hours).

### II.3.2. Inducing Factors (Positive Controls)

During bibliographic searches, 2 inducers have been identified as being able to induce the synthesis of TAFA4 at the cutaneous level: LPS and UVs (Hoeffel et al., 2021; Wang et al., 2015).

### II.3.3. Case of LPS Stimulation

Pretreatment of monocytes/macrophages with PMA (Phorbol 12-myristate 13-acetate, 10 ng/ml) for 24 hours and then stimulated with LPS (1 μg/ml) for 12 hours or 24 hours (Wang et al., 2015).

### II.3.4. Case of UV Stimulation

After removal of the culture medium and rinsing with D-PBS, the cells are irradiated with UVs.

Data on macrophage exposure protocols described:
at a dose of 6.7 J/cm² of UVA for 15 minutes in a Biosun System comprising an irradiation chamber equipped with a 365 nm illumination lamp, a dosimeter/sensor, a calibrator and software (Shen et al., 2014); or
UVAs by a black lamp with a peak emission energy at 360 nm. The emitted dose has been measured by a radiometer (UVX-36; UVP, Inc., San Gabriel, CA). No UVB has been detected with a UVX-31 sensor. The irradiance at the sample level is 2.5 mW/cm² (Kawano et al., 2015).

### II.4 Selection of Compounds or Compositions to be Evaluated

Different molecules can be preselected and tested. These molecules will be directly solubilised in the culture medium or solubilised in ethanol or DMSO according to their nature.

### II.5 MTT Cell Viability Test

See point 1.7.

### II.6 Quantification of the TAFA4 Expression by RT-qPCR

After 30 minutes to 24 hours of incubation with the active agents, an extraction of the total RNAs is performed using the Rneasy Plus Mini Kit according to the recommendations of the supplier (Qiagen).

The assay of total RNAs makes it possible to know the quantity and quality of RNAs in the cell extract. The assay is performed using the Bioanalyser and the RNA 6000 Nano kit.

Reverse Transcription performed using the QuantiTect Reverse Transcription kit (Qiagen) makes it possible to obtain cDNA.

A real-time Quantitative PCR at each treatment time is performed for the TAFA4 gene. The Qiagen QuantiFast SYBR Green PCR Kit is used.
Housekeeping gene: GAPDH.
Gene of interest: TAFA4→see the specific primers of the gene

### II.7 Assay of TAFA4 in Culture Supernatants by ELISA

The quantification of TAFA4 in supernatants is done thanks to an ELISA test, Enzyme Linked Immunosorbent Assay (Ref: abx522987, Cliniscience, France) with the reagents recommended by the manufacturer.

The Optical Density (OD) is read with a spectrophotometer at 450 nm. The concentration of TAFA4 is calculated from the standard range (0.1561 ng/ml to 10 ng/ml) established with the recombinant human TAFA4 protein. The results are expressed in ng/ml of TAFA4 per well. On the cell layer, an MTT viability test is performed. TAFA4 assays are used, only if cell viability is greater than 80%, and are related to this cell viability value.

### II.8 Statistical Analyses

The data have been collected from independent experiments performed in triplicate. Quantitative analyses are expressed in the form of mean±standard deviation. Statistical significance is determined by a Student test. The differences are considered as statistically significant from p<0.05.

(NS: p>0.05; *: p<0.05; **: p<0.01; ***: p<0.001).

### Example III—Differentiation of SH-SY5Y Cells into Sensory Neuronal Cells and Analysis of TAFA4 Levels

### III.1 Introduction

The aim of the study is to test the modulation of TAFA4 expression in a neuronal cell type able to produce it.

### III.2 Materials and Methods

### III.2.1 SH-SY5Y Cell Culture and Differentiation

The SH-SY5Y human neuroblastoma cells used in this study are obtained from Sigma Aldrich (94030304). The cells are thawed at P13 in a water bath at 37° C., then immediately placed in DMEM. The cell suspension is centrifuged at 125×g for 7 minutes at 4° C. and the pellets are suspended in a mixture of 1:1 Eagle's Minimum Essential Medium, and F12 Medium, supplemented with 10% faetal bovine serum, and with a PS solution.

The cells are seeded in the 96-well plate at a density of 7,000 cells per well and are cultured at 37° C. in an incubator at 95% air, 5% CO₂. To avoid any edge effect, the first and the last column, as well as the first and the last line of the plate are not used in the study. Empty wells are filled with water. Cells are left to grow to confluence (80%).

At confluence (1 day later), the cells develop in a mixture of 1:1 DMEM medium, and F12 Medium, supplemented with 1% faetal bovine serum, NGF (5 ng/ml), PS solution (1%) and 10 μM retinoic acid (RA), to enable their differentiation. The medium was changed every 2 days. For 96-well plates, only 60 wells were used.

III.2.2 Expression/Release of TAFA4 at Different Times, D8, D10, D12, after Exposure to Compounds

After 8, 10 or 12 days of differentiation with RA, the cells are stressed for 24 hours with two compounds individually diluted at different concentrations in the culture medium. At the same time, a non-stressed control condition is performed at each differentiation time (8, 10 and 12 days; reference values).

Only 100 μL of culture medium is applied.

III.2.3 qPCR: TAFA4 Gene Expression

Several hours (according to the stressors and steps) after the application of the stressor, the cells are lysed with the RNS XS nucleo spin kit (Macherey Nagel). Briefly, 100 μL of lysis buffer is added per well, according to the instructions of the manufacturer (Macherey Nagel). The procedure comprises a DNase treatment. RNA extraction is performed in a laboratory room dedicated to biomolecular experiments, with sterile, DNase/RNase-free solutions and laboratory consumables (for example, filtered tips, tubes). The total quantity and purity of the RNA present in each sample is evaluated by spectrophotometry using a Nanodrop device (NanoDrop technologies LLC). The RNA samples are preserved at a temperature below −70° C. The cDNAs are obtained by reverse transcription using prime script reverse transcriptase (SensiFAST cDNA synthesis kit, Bioline meridian) from 25 ng of total RNA according to the instructions of the manufacturer. The cDNA samples are preserved at −15/−25° C. The PCR reaction is achieved using the CFX96™ real-time system (Biorad). An equivalent of 10 ng of initial RNA is subjected to PCR amplification by using 3 μM primers and SYBR Premix (ONEGreen Fast qPCR Premix, OZYME). The PCR reaction is achieved by using the following cycle procedure: 95° C. for 30s, then 95° C. for 5s, 60° C. for 30s, and a total of 40 cycles is performed.

Each sample is analysed for the selected gene (GAPDH and TAFA4) using RT-qPCR to measure the relative expression levels of mRNAs encoding for the selected genes. The analysis of gene expression is evaluated using the 2-ΔΔCt method (Livak and Schmittgen, 2001), where Ct is the threshold value of the cycle. A negative control (without cDNA) is performed for each pair of primers. The expression is standardised against a housekeeping gene (for example, GAPDH).

### III.2.4 Statistical Analyses

All values are expressed as a mean±SEM (standard error of the mean). The statistical analysis is performed by one-way ANOVA, followed by Fisher's LSD test. p<0.05 is considered significant.

### III.3 Results and Conclusions

These tests have made it possible to identify modulators, i.e. inhibitors and inducers of TAFA4 expression in SH-SY5Y cells.

### Example IV—Evaluation of the Effect of Compounds on TAFA4 Synthesis in Normal Human Fibroblast Cultures

### IV.1 Introduction

The aim of the study is to develop a cell model, making it possible to quantify TAFA4 synthesis in skin cells.

### IV.2 Materials and Methods

### IV.2.1 Cell Culture

Normal human fibroblasts (NHFs) have been used in this study (Lonza-Switzerland). The cells are seeded at 10,000 cells in 24-well plates in DMEM+1% Penicillin-streptomycin (P/S). After 24 hours of incubation, the cells were incubated with the compounds presented in table 1.

TABLE 1

| Compound | | Product and supplier |
|---|---|---|
| tested | Concentration | references |
| Valproic acid | 0.025% = 0.25 mg/ml; | PHR1061, SIGMA |
| | 0.05% = 0.5 mg/ml | |
| TGF-β | 10 ng/ml, 100 ng/ml | 100-21, PEPROTECH |
| LPS | 1 μg/ml | L2880-10MG, SIGMA |

After 72 hours of incubation at 37° C., 5% CO₂, the supernatants and protein extracts (obtained from the cell layer) are recovered and stored at −80° C.

Before assaying the TAFA4 protein, an evaluation of the toxicity of the compounds tested is performed using the MTT test. Only the concentrations which make it possible to obtain a viability greater than 80% are selected to be quantified by ELISA and dot blot.

The data have been collected from independent experiments performed in triplicate.

### IV.2.2 MTT Cell Viability Test

See point 1.7.

### IV.2.3 TAFA4 Array by Dot Blot

Dot blotting is an immunoenzymatic detection method for quantifying a protein in a sample after it has been deposited on a nitro-cellulose membrane in a well. Specific equipment (Biorad, France) is used with a nitrocellulose membrane. The equipment and consumables used in this experiment are indicated in table 2 below.

TABLE 2

| Product | Reference | Supplier |
|---|---|---|
| Dot blot | Bio dot microfiltration apparatus, | Biorad |
| | ref.: 170-6545 | |
| Nitrocellulose | NITROCEL MEMB.45, 7 × 8.5 cm, | Biorad |
| membrane | 10PK, ref.: 1620145 | |
| Vacuum pump | KNF Lab Laboport, ref.: N86KN.18 | Dutscher |
| Trisma base | T6066-500G | Sigma |
| NaCl | S9625-500G | Sigma |
| Standard | Recombinant human TAFA4/FAM19A4 | R&D system |
| TAFA4 | protein, ref.: 5099-TA-050 | (Biotechne) |
| Skimmed milk | Regilait, Skimmed milk, ref.: L MC3 | Carrefour |
| Tween 20 | P7949-100ML | Sigma |
| Primary | Human TAFA4/FAM19A4 Mab | R&D system |
| antibody | (Clone 480103), ref.: MAB5099 | (Biotechne) |
| Secondary | GOAT ANTIMOUSE IGG (H + L), | Thermo Fisher |
| antibody | ref.: 10799354 | |
| ECL | Clarity Western ECL Subs, | Biorad |
| | 200 ml, ref.: 1705060 | |

The device is subjected to a vacuum pressure which will make it possible to create the suctioning of the sample (supernatant or protein extract) and therefore, the binding of proteins to the nitrocellulose membrane. The equipment is placed at a height with respect to the vacuum pump, to respect the principle of gravity.

The membrane is previously hydrated with 100 μl of TBS buffer (Tris base+NaCl diluted in water adjusted to pH 7.5) per well. This operation is repeated twice in order to optimise the hydration of the membrane for optimal binding of the proteins contained in the samples. The samples to be analysed (supernatants and protein extracts) and the different solutions of TAFA4 recombinant proteins at different concentrations of the TAFA4 standard range (0.78 to 100 ng/ml) are deposited in the plate (volume 100 μl/well used for the protein extracts and 500 μl/well-used for the supernatants) then the suction flow is activated. When all of the samples are suctioned, 100 μl of TBS is added per well to ensure that all of the sample is passed over the membrane. The proteins contained in the samples will thus be deposited on the nitrocellulose membrane and create a spot of 1 cm².

For marking and revealing, a step of saturating the non-specific sites is carried out by incubating the membrane in a bath containing 5% of skimmed milk in a TBS buffer (volume: 30 ml) under stirring at 4° C. during the night. The next day, the membrane was washed with 20 ml of TBS-Tween buffer (TBS+Tween 20 at 0.05%) under stirring for 10 minutes. This washing is repeated 3 times, by changing the washing buffer (volume: 30 ml). The membrane is incubated in a bath containing the primary antibody diluted to 1/200 in TTBS buffer+1% skimmed milk (volume: 20 ml) for 2 hours at ambient temperature under stirring. Then, the membrane is washed in the same way as described above.

The membrane is then incubated in a solution containing the secondary antibody (antibody coupled to HRP) diluted to 1/500 in TTBS buffer+1% skimmed milk (volume: 20 ml) for 2 hours at ambient temperature under stirring. The membrane is washed in the same way as described above. The secondary antibody alone is used as a negative control.

The proteins present in each of the deposits are revealed by incubating the membrane in a bath of pure ECL, a chemiluminescent substrate for detecting the activity of peroxidase (HRP, horseradish peroxidase) conjugated to the secondary antibody. The presence of the TAFA4 protein on the membrane is conveyed by the formation of a grey to black spot depending on the intensity of the reaction and therefore on the quantity of protein. The membrane is read with the Fusion FX device (Vilber Lourmat) coupled with the VisionCapt software (Vilber Lourmat). The quantification of the TAFA4 protein in the samples tested is done with the VisionCapt software which makes it possible to measure the intensity of the various spots. The quantity of samples is then produced using the standard curve of the TAFA4 standard established with the recombinant protein

### IV.2.4 Statistical Analyses

The data have been collected from independent experiments performed in triplicate. Quantitative analyses are expressed in the form of mean±standard deviation. Statistical significance is determined by a Student test. The differences are considered as statistically significant from p<0.05.

(NS: p>0.05; *: p=0.05; **: p=0.01; ***: p=0.001).

### IV.3 Results and Conclusions

FIG. 1 shows the effect of valproic acid, TGF-β and LPS on the modulation of TAFA4 in the NHF supernatant after 72 hours of incubation. The results are expressed as a percentage of TAFA4 expression by standardising the control to 100% (DMEM medium control). The data have been obtained from 2 independent experiments performed in triplicate.

Valproic acid and TGF-β induce the synthesis of TAFA4 in a dose-dependent manner (statistically significant induction for 2 out of 2 doses for TGF-β; 1 out of 2 doses for valproic acid).

LPS inhibits TAFA4 synthesis (statistically significant inhibition) at the dose tested.

### Example V—Evaluating the Effect of Compounds on TAFA4 Synthesis in Normal Human Keratinocyte Cultures

### V.1 Introduction

The aim of the study is to develop a cell model, making it possible to quantify TAFA4 synthesis in skin cells.

### V.2 Materials and Methods

### V.2.1 Cell Culture

Normal human keratinocytes (NHK) have been used in this study. They come from Promocell (Germany). The cells are seeded at 100,000 cells/well in the 24-well plates in KGM. After 24 hours, the cells are incubated with the compound shown in the table below in KBM+P/S medium at 37° C., 5% CO₂.

After 72 hours of incubation, the supernatants and protein extracts (obtained from the cell layer) were recovered and stored at −80° C. The test compound is indicated in table 3 below.

TABLE 3

| | | Product and supplier |
|---|---|---|
| Compound tested | Concentration | references |
| EGF | 30 ng/ml | AF-100-15, Peprotech |

After 72 hours of incubation at 37° C., 5% CO₂, the supernatants and protein extracts (obtained from the cell layer) are recovered and stored at −80° C.

Before assaying the TAFA4 protein, an evaluation of the toxicity of the compounds is performed using the MTT test. Only the concentrations making it possible to obtain a viability greater than 80% are selected to be quantified by ELISA and dot blot.

The data have been collected from independent experiments performed in triplicate.

### V.2.2 MTT Cell Viability Test

See point I.7.

### V.2.3 TAFA4 Array by Dot Blot

See point IV.2.3.

### V.2.4 Statistical Analyses

See point IV.2.4.

### V.3 Results and Conclusions

FIG. 2 shows the effect of EGF on the modulation of TAFA4 in the NHK supernatant after 72 hours of incubation. The results are expressed as a percentage of TAFA4 expression by standardising the control to 100% (KGM medium control). The data have been obtained from an independent experiment carried out in triplicate. EGF tested at 30 ng/ml inhibits the synthesis of TAFA4 (statistical significance).

### BIBLIOGRAPHY

Atef A., El-Rashidy M. A., Abdel Azeem A., Kabel A. M. (2019) The Role of Stem Cell Factor in Hyperpigmented Skin Lesions. Asian Pac J Cancer Prev.; 20 (12): 3723-3728.
Delfini, M.-C., Mantilleri, A., Gaillard, S., Hao, J., Reynders, A., Malapert, P., Alonso, S., François, A., Barrere, C.,
Seal, R., et al. (2013). TAFA4, a chemokine-like protein, modulates injury-induced mechanical and chemical pain hypersensitivity in mice. Cell Rep 5:378-388.
Grabbe J., Welker P., Dippel E., Czarnetzki B. M. (1994) Stem cell factor, a novel cutaneous growth factor for mast cells and melanocytes. Arch Dermatol Res. 287:78-84.
Hoeffel G, Debroas G, Roger A, Rossignol R, Gouilly J, Laprie C, Chasson L, Barbon P V, Balsamo A, Reynders A, Moqrich A, Ugolini S. (2021). Sensory neuron-derived TAFA4 promotes macrophage tissue repair functions. Nature 594:94-99.
Jung M, Dourado M, Maksymetz J, Jacobson A, Laufer B I, Baca M, Foreman O, Hackos D H, Riol-Blanco L, Kaminker J S. (2023) Cross-species transcriptomic atlas of dorsal root ganglia reveals species-specific programs for sensory function. Nat Commun. 14:1-366.
Huet F, Dion A, Batardière A, Nedelec A S, Le Caër F, Bourgeois P, Brenaut E, Misery L. (2018) Sensitive skin can be small fibre neuropathy: results from a case-control quantitative sensory testing study. Br J. Dermatol. 179:1157-1162.
Horiba, S., Kami, R., Tsutsui, T., and Hosoi, J. (2022). IL-34 Downregulation-Associated M1/M2 Macrophage Imbalance Is Related to Inflammaging in Sun-Exposed Human Skin. JID Innov 2:100112.
Kambrun, C., Roca-Lapirot, O., Salio, C., Landry, M., Moqrich, A., and Le Feuvre, Y. (2018). TAFA4 Reverses Mechanical Allodynia through Activation of GABAergic Transmission and Microglial Process Retraction. Cell Rep 22:2886-2897.
Kawano, A., Hayakawa, A., Kojima, S., Tsukimoto, M., and Sakamoto, H. (2015). Purinergic signaling mediates oxidative stress in UVA-exposed THP-1 cells. Toxicol Rep 2:391-400.
Leeman, A., Jenkins, D., Del Pino, M., Ordi, J., Torne, A., Doorbar, J., Meijer, C. J. L. M., van Kemenade, F. J., and Limat A, Hunziker T. (2002) Use of epidermal equivalents generated from follicular outer root sheath cells in vitro and for autologous grafting of chronic wounds. Cells Tissues Organs. 172:79-85.
Livak K J, Schmittgen T D. (2001) Analysis of relative gene term data using real-time quantitative PCR and the 2 (−Delta C (T)) Method. Methods. 25 (4): 402-8
Liu, D., Peng, H., Sun, Q., Zhao, Z., Yu, X., Ge, S., Wang, H., Fang, H., Gao, Q., Liu, J., et al. (2017). The Indirect Efficacy Comparison of DNA Methylation in Sputum for Early Screening and Auxiliary Detection of Lung Cancer: A Meta-Analysis. Int J Environ Res Public Health 14: E679.
Misery L., Ständer S., Szepietowski J. C., Reich A., Wallengren J, Evers A. W., Takamori K., Brenaut E., Le Gall-Ianotto C., Fluhr J., Berardesca E., Weisshaar E. (2017) Definition of Sensitive Skin: An Expert Position Paper from the Special Interest Group on Sensitive Skin of the International Forum for the Study of Itch. Acta Derm Venereol. 97:4-6.
Misery L, Jourdan E, Huet F, Brenaut E, Cadars B, Virassamynaik S., Sayag M, Taieb C (2018a) Sensitive skin in France: a study on prevalence, relationship with age and skin type and impact on quality of life. J Eur Acad Dermatol Venereol. 32:791-795.
Misery L, Jourdan E, Abadie S, Ezzedine K, Brenaut E, Huet F, Sayag M, Taieb C (2018b). Development and validation of a new tool to assess the Burden of Sensitive Skin (BoSS). J Eur Acad Dermatol Venereol. 12:2217-2223.
Poumay Y, Dupont F, Marcoux S, Leclercq-Smekens M, Hérin M, Coquette A (2004) A simple reconstructed human epidermis: preparation of the culture model and use in vitro studies. Arch Dermatol Res. 296:203-11.
Qiu S, Luo X, Mo L, Zhang S, Liao Y, Guan L, Yang L, Huang Q, Liu D, Yang P. (2022) TAFA4-IL-10 axis potentiate immunotherapy for airway allergy by induction of specific regulatory T cells. npj Vaccines 7, 133.
Quint, W. G. V. (2020). Expression of p16 and HPV E4 on biopsy samples and methylation of FAM19A4 and miR124-2 on cervical cytology samples in the classification of cervical squamous intraepithelial lesions. Cancer Med 9:2454-2461.
Salio, C., Aimar, P., Malapert, P., Moqrich, A., and Merighi, A. (2021). Neurochemical and Ultrastructural Characterization of Unmyelinated Non-peptidergic C-Nociceptors and C-Low Threshold Mechanoreceptors Projecting to Lamina II of the Mouse Spinal Cord. Cell Mol Neurobiol 41:247-262.
Shen, C., Turney, T. W., Piva, T. J., Feltis, B. N., and Wright, P. F. A. (2014). Comparison of UVA-induced ROS and sunscreen nanoparticle-generated ROS in human immune cells. Photochem Photobiol Sci 13:781-788.
Ständer, S., Schneider S. W., Weishaupt, C., Luger, T. A., and Misery, L. (2009) Putative neuronal mechanisms of sensitive skin Exp. Dermatol., 18:417 423.
Tom Tang, Y., Emtage, P., Funk, W. D., Hu, T., Arterburn, M., Park, E. E. J., and Rupp, F. (2004). TAFA: a novel secreted family with conserved cysteine residues and restricted term in the brain. Genomics 83:727-734.
Wang, W, Li, T., Wang, X, Yuan, W, Cheng, Y, Zhang, H, Xu, E., Zhang, Y., Shi, S, Ma, D, et al. (2015) FAM19A4 is a novel cytokine ligand of formyl peptide receptor 1 (FPR1) and is able to promote the migration and phagocytosis of macrophages. Cell Mol Immunol 12, 615-624.
Yoo S, Santos C, Reynders A, Marics I, Malapert P, Gaillard S, Charron A, Ugolini S, Rossignol R, El Khallouqi A., Springael J Y, Parmentier M, Saurin A J, Goaillard J M, Castets F, Clerc N, Moqrich A (2021) TAFA4 relives injury-induced mechanical hypersensitivity through LDL receptors and modulation of spinal A-type K+ current. Cell Rep 37, 109884.

1. A method for the selection of at least one compound intended for the prevention and/or care of sensitive and/or sensitized skin, implementing the identification of a modulation of the content of TAFA4 and/or nucleic acid.

2. The method according to claim 1, wherein said method comprises the following steps:
a) Bringing said at least one compound into contact with at least one skin cell;
b) Measuring the content of TAFA4 and/or of nucleic acid encoding TAFA4;
c) Comparing the value obtained in step b) with a reference value;
d) Selecting said at least one compound inducing an increase in the content of TAFA4 and/or of nucleic acid in step c).

3. The method according to claim 2, wherein said reference value is the measurement obtained without bringing said at least one skin cell into contact with said at least one compound.

4. The method according to claim 2, wherein the said content of TAFA4 and/or nucleic acid encoding TAFA4 is measured in the intra- and/or extracellular medium of said at least one skin cell.

5. The method according to claim 2, wherein:
an increase in the content of TAFA4 corresponds to at least 120% with respect to said reference value; and/or
an increase in the content of nucleic acid encoding TAFA4 corresponds to at least 120% with respect to said reference value.

6. The method according to claim 1, wherein the at least one compound is not the TAFA4 protein or one of its biologically active derivatives.

7. The method according to any one of the preceding claim 1, wherein said method is an in vivo or ex vivo method and in that the bringing into contact of step a) is performed by topical application of said at least one compound to the skin of a whole living organism, a skin explant, a reconstructed epidermis or a reconstructed skin.

8. The method according to claim 2, wherein:
this is an in vitro method,
said at least one skin cell is chosen from keratinocytes, melanocytes, sebocytes, Merkel cells, vascular smooth muscle cells, specialised muscle cells, fibroblasts, mastocytes, lymphocytes, monocytes, dendritic cells; Langerhans cells, adipocytes, nerve cells, macrophages; endothelial cells; and mixtures thereof; and
said bringing into contact of step a) is performed by adding into the culture medium.

9. The method according to claim 8, wherein the at least one skin cell is:
at least one macrophage; and/or
at least one keratinocyte; and/or
at least one fibroblast; and/or
at least one nerve cell.

10. The method according to claim 2, wherein step b) of measuring the content of TAFA4 and/or of nucleic acid, encoding TAFA4 is carried out at a protein level by an ELISA test, immunohistochemistry, colorimetric method, western blot, dot blot or western simplified by capillary immunoelectrophoresis (WES) and/or at the nucleic level by in situ hybridisation, northern blot, sequencing or RT-PCR.

11. The method according to claim 2, wherein, before the implementation of step a), said at least one skin cell has been brought into contact, by topical application or by adding into the culture medium, with a sensitising agent.

12. The method according to claim 1, wherein:
the amino acid sequence of TAFA4 corresponds to the sequence SEQ ID NO: 1 and/or to SEQ ID NO: 2 and/or to a sequence identical to one of the sequences SEQ ID NO: 1 to 2 to at least 60%; and/or
the nucleotide sequence of TAFA4 corresponds to the sequence SEQ ID NO: 3, SEQ ID NO: 4, SEQ ID NO: 5, SEQ ID NO: 6 or SEQ ID NO: 7 or to a sequence identical to one of the sequences SEQ ID NO: 3 to 7 to at least 60%.

13. A method of formulating a cosmetic and/or food composition, non-therapeutic, comprising the incorporation into said composition of at least one compound selected by the method according to claim 1.

14. The method according to claim 1, wherein the compound is ecobiological.

15. The method according to claim 1, wherein the modulation is an increase of the content of TAFA4 and/or nucleic acid encoding TAFA4.

16. The method according to claim 1, wherein the modulation is an increase of the content of messenger ribonucleic acid (mRNA) encoding TAFA4.

17. The method according to claim 13, wherein the cosmetic and/or food composition, non-therapeutic is ecobiological.

18. The method according to claim 13, wherein the cosmetic and/or food composition, non-therapeutic is in a galenic form suitable for topical and/or oral administration.
